(19) United States
(12) Patent Application Publication
(10) Pub. No.: US 2019/0270686 A1
(43) Pub. Date: Sep. 5, 2019

(54) ETHYLENE RECOVERY AND PURIFICATION

(71) Applicant: SABIC Global Technologies B.V., Bergen op Zoom (NL)

(72) Inventors: Te Chang, Sugar Land, TX (US); Saud Abdullah R. Alhagbani, Thuwal (SA); Sanjeev Rao, Sugar Land, TX (US)

(21) Appl. No.: 16/333,731

(22) PCT Filed: Sep. 7, 2017
(86) PCT No.: PCT/IB2017/055393

§ 371 Date: Mar. 15, 2019

Related U.S. Application Data
(60) Provisional application No. 62/395,010, filed on Sep. 15, 2016.

(51) Int. Cl. C07C 5/09; C07C 7/04; C07C 7/11; C07C 7/00

(52) U.S. Cl. CPC C07C 5/09; C07C 7/04; C07C 7/11; C07C 2523/06; C07C 2523/42; C07C 2523/60; C07C 7/005

(57) ABSTRACT

A method for recovering ethylene from cracked gas comprises: (a) hydrogenating a composition comprising acetylene and an acetylene extraction solvent to produce a first gaseous hydrocarbon stream and a first liquid stream; (b) contacting the first gaseous hydrocarbon stream with a second acetylene extraction solvent stream in an acetylene scrubber unit to produce a second liquid stream and a gaseous ethylene product stream; (c) contacting the first liquid stream of step (a) with the second liquid stream of step (b) in an ethylene stripper unit to produce a recovered gaseous ethylene stream and a third liquid stream; and (d) providing the recovered gaseous ethylene stream to step (b).

### CROSS-REFERENCE TO RELATED APPLICATIONS

This application claims the benefit of priority of U.S. Provisional Patent Application No. 62/395,010 filed Sep. 15, 2016, which is hereby incorporated by reference in its entirety.

### TECHNICAL FIELD

The present invention relates to processes that remove acetylene and recovers ethylene from cracked gas streams in ethylene production plants.

### BACKGROUND OF THE INVENTION

Ethylene (C₂H₄) is a common building block for a variety of petrochemicals. One way of producing ethylene is to steam crack hydrocarbon feedstocks such as naphtha, natural gas liquids, ethane, and propane. In the steam cracking (pyrolysis) process, the hydrocarbons are superheated in a reactor to temperatures as high as 750-950° C. In addition to ethylene, the cracking process can produce other hydrocarbons. Those other hydrocarbons may include acetylene (C₂H₂). Typically, the amount of acetylene produced by the cracking process is small in relation to the amount of ethylene and other hydrocarbon products. For example, a cracked gas in an ethylene production plant can include 0.3 wt. % to 0.9 wt. % acetylene, but the amount of acetylene produced may increase beyond this with increase in cracking severities and heavier feedstocks. Notwithstanding the seemingly small amounts of acetylene in cracked gas, recovery of ethylene from the cracked gas typically requires the removal of most, if not all, of the acetylene.

Such removal of acetylene from cracked gas is influenced by the product specifications of polymer-grade ethylene. The product specifications for polymer grade ethylene are stringent, allowing for very little contaminants to be present in the product. These stringent product specifications are dictated, at least in part, by the processes in which ethylene is used as raw material for forming other petrochemicals. For example, ethylene is used in a polymerization process to form polyethylene. But acetylene has a detrimental effect on the polymerization process, even when the acetylene is at parts per million (ppm) levels in ethylene product streams used as raw material.

Polymer-grade ethylene is typically sold as a product stream having a minimum ethylene content of 99% and a maximum acetylene content of 5 ppm. To achieve ethylene of 99% or as much as 99.9% purity, the ethylene industry has primarily used selective hydrogenation to remove acetylene from cracked gas. In the hydrogenation process of conventional ethylene production processes, a gas phase acetylene converter and an acetylene polishing reactor are used at either the front end or the back end of C₂ separation steps. In some gas-to-ethylene processes, a back-end acetylene polishing reactor is designed to mitigate acetylene slip.

In conventional methods of ethylene production, at the front end of separating C₂ streams from the cracked gas (using deethanizer or depropanizer), the cracked gas may be subjected to vapor phase catalytic hydrogenation (e.g., as disclosed in U.S. Pat. No. 8,309,776 to van Egmond et al.). Catalytic hydrogenation can also be applied at the back end of separating C₂ streams from the cracked gas, after the cold-box, for product ethylene purification. Typically, the hydrogenation reaction takes place in the vapor phase over palladium (Pd) catalysts. However, vapor phase selective hydrogenation processes using palladium catalysts have narrow operating temperature windows and high runaway potentials, at high hydrogen concentration and variable CO concentration in the cracked gas feed.

Solvent extraction of acetylene, using organic solvent such as dimethyl formamide (DMF), N-methyl-2-pyrrolidone (NMP), acetone, methanol, and water mixtures is known in the art (e.g., as disclosed in U.S. Pat. No. 3,004,629 to Cottle. However, because most commercial ethylene plants generate a relatively low amount of acetylene, solvent extraction is seldom used for acetylene removal from these plants. Instead, most ethylene plants remove acetylene from ethylene streams by selective hydrogenation.

The methods described above are typically geared towards acetylene removal for ethylene produced from ethane, natural gas liquids (NGLs), and naphtha pyrolysis and are not particularly suitable for ethylene streams produced by gas-to-ethylene technology. Examples of gas-to-ethylene processes involving the production of ethylene directly from natural gas are described in U.S. Pat. No. 7,183,451 to Gattis et al., and U.S. Pat. No. 7,915,466 to Gattis et al. Gas-to-ethylene processes involve converting natural gas to liquid hydrocarbons via combustion and pyrolysis. In these processes, the cracked gas is typically rich in acetylene, which can be separated and converted to ethylene. Cracked gas generated by gas-to-ethylene technology typically has high acetylene concentration (e.g., ethylene concentration ranging from 5 to 15 vol. %), which may increase with increase of heavier hydrocarbons in the natural gas feedstock. At such relatively high acetylene concentrations, conventional vapor phase hydrogenation technology provides poor ethylene yield. U.S. Pat. No. 8,460,937 to Johnson et al. describes that ethylene yield up to 96% can be achieved by liquid phase acetylene hydrogenation using N-methyl-2-pyrrolidone as solvent. U.S. Pat. No. 8,013,197 to Peterson et al. describes a multi-stage flashing process for ethylene recovery commonly used in conventional ethylene processes. Processes that produce crude ethylene via consecutive flashings of hydrogenation reactor effluent can result in the crude ethylene containing an excessive amount of unreacted acetylene, which, in turn, causes ethylene product from the crude ethylene to not meet polymer-grade ethylene product specifications. Moreover, processes that produce crude ethylene via consecutive flashings of hydrogenation reactor effluent may only achieve 65 to 80% ethylene recovery.

Referring to FIG. 1, a prior art gas-to-ethylene system for acetylene hydrogenation and removal from cracked gas is shown. The system includes multi-stage flashing tanks (flash tanks 111 and 115). Cracked gas stream 100 can be split into two fractions, stream 101 and stream 102. Stream 101 can be fed to acetylene absorber column 105. Stream 102 can bypass acetylene absorber column 105 and be fed directly into hydrogenation reactor 110. Acetylene absorber column 105 recovers ethylene and acetylene from stream 101. Acetylene extraction solvent stream 124 can be fed overhead to acetylene absorber column 105. Absorber overhead vent gas 103 can flow from the top of acetylene absorber column 105. Solvent stream 104 from the bottom of acetylene absorber column 105 can be preheated, using preheater 106. Subsequently, solvent stream 104 is combined with stream 102 to form mixed stream 107, which is fed concurrently into hydrogenation reactor 110. At the bottom of hydrogenation reactor 110 effluent reservoir, reactor effluent vapor stream 109 and hydrogenation reactor liquid stream 108 disengages. Hydrogenation reactor liquid stream 108 can be depressurized to produce vapor stream 112. Vapor stream 112 can mix with reactor effluent vapor stream 109 to form mixed hot vapor stream 113. Mixed hot vapor stream 113 can be cooled by air-fan heat exchanger 131 and flowed to flash vessel 115 to knock out entrained solvent. Knock-out liquid stream 114 from flash tank 111 can be cooled by air-fan heat exchanger 132. Knock-out liquid stream 114 and knock-out liquid stream 116 are collected in solvent accumulation vessel 120. Vapor stream 117 from flash vessel 115 can be combined with the vapor stream 121 from solvent accumulation vessel 120 to form crude ethylene product stream 122. A fraction of stream 123 cam be diverted as stream 126 to solvent regeneration column 125 where a fraction of light hydrocarbons can be purged in overhead vapor stream 127 and in condensed liquid stream 128. Bottom stream 129 flows from solvent regeneration column 125 and can be routed to solvent accumulation vessel 120. Makeup fresh solvent 118 is added to acetylene extraction solvent 124.

Some conventional two-stage flashing processes such as the one illustrated in FIG. 1 withhold 20 to 30% ethylene produced in the recycle solvent, which is then stripped away into recycle fuel gas at the cracked gas absorber overhead. This can lead to a significant reduction in ethylene yield while the crude ethylene off gas may be contaminated with unreacted acetylene up to thousands ppm of acetylene in the ethylene end-product, thereby thwarting the production of polymer-grade ethylene.

### SUMMARY OF THE INVENTION

A discovery has been made that provides a solution to the aforementioned deficiencies that are typically seen in processes involving removal of acetylene from ethylene product streams. The solution is premised, in part, on a process for recovering ethylene from cracked gas that includes acetylene scrubbing and ethylene stripping of streams that can include ethylene. The process may be particularly suitable in applications that involve gas-to-ethylene technology that produces ethylene by combustion pyrolysis of methane, natural gas, and/or shale gas that forms acetylene as an intermediary product. Embodiments of the invention include implementing acetylene scrubbing and ethylene stripping operations into a liquid phase acetylene hydrogenation technology using solvents such as N-methyl-2-pyrrolidone and/or dimethyl formamide (DMF).

Embodiments of the invention include a process for purifying and recovering ethylene from a cracked gas stream. The process can include step (a): hydrogenating a composition that includes acetylene and an acetylene extraction solvent in a hydrogenation unit under conditions sufficient to produce: (1) a first gaseous hydrocarbon stream comprising ethylene and acetylene and (2) a first liquid stream that includes ethylene and acetylene gases dissolved in the acetylene extraction solvent. The first liquid stream can include more acetylene than the first gaseous hydrocarbon stream. The process can also include step (b): contacting the first gaseous hydrocarbon stream with a second acetylene extraction solvent stream in an acetylene scrubber unit under contacting conditions sufficient to extract a portion of the acetylene from the gaseous hydrocarbon stream and produce: (i) a second liquid stream that includes ethylene and acetylene gases dissolved in the acetylene extraction solvent, and (ii) a gaseous ethylene product stream. The process may further include step (c): contacting the first liquid stream of step (a) with the second liquid stream of step (b) in an ethylene stripper unit under separating conditions sufficient to produce a recovered gaseous ethylene stream and a third liquid stream that can include acetylene dissolved in the acetylene extraction solvent. The process can also include (d) providing the recovered gaseous ethylene stream to step (b) to provide further removal of acetylene, if present, from the recovered gaseous ethylene stream. In the context of the current invention, 20 embodiments are described. Embodiment 1 includes a process for purifying and recovering ethylene from a cracked gas stream, the process comprising: (a) hydrogenating a composition comprising acetylene and an acetylene extraction solvent in a hydrogenation unit under conditions sufficient to produce: a first gaseous hydrocarbon stream comprising ethylene and acetylene; and a first liquid stream comprising ethylene and acetylene gases dissolved in the acetylene extraction solvent, wherein the first liquid stream comprises more acetylene than the first gaseous hydrocarbon stream; (b) contacting the first gaseous hydrocarbon stream with a second acetylene extraction solvent stream in an acetylene scrubber unit under contacting conditions sufficient to extract a portion of the acetylene from the gaseous hydrocarbon stream and produce: a second liquid stream comprising ethylene and acetylene gases dissolved in the acetylene extraction solvent, and a gaseous ethylene product stream; (c) contacting the first liquid stream of step (a) with the second liquid stream of step (b) in an ethylene stripper unit under separating conditions sufficient to produce a recovered gaseous ethylene stream and a third liquid stream comprising acetylene dissolved in the acetylene extraction solvent; and (d) providing the recovered gaseous ethylene stream to step (b). Embodiment 2 is the process of embodiment 1, wherein the gaseous ethylene product stream comprises 1 ppm or less of acetylene, and/or the ethylene recovery efficiency is at least 80%, preferably 100%. Embodiment 3 is the process of any one of embodiments 1 to 2, wherein the contacting conditions of step (b) and the separating conditions of step (c) comprise a pressure in the acetylene scrubber unit and a pressure in the ethylene stripper unit, and wherein the pressure in the ethylene stripper unit is less than the pressure in the acetylene scrubber unit. Embodiment 4 is the process of embodiment 3, wherein the acetylene scrubber unit pressure is from 0.01 MPa(g) to 0.5 MPa(g), preferably 0.03 MPa(g) to 0.5 MPa(g), with the ethylene stripper unit pressure preferably 0.01 MPa(g) to 0.4 MPa(g) less than the acetylene scrubber unit pressure. Embodiment 5 is the process of any one of embodiments 1 to 4, wherein the acetylene is produced by partial oxidation of methane, methane pyrolysis, or heating a composition of methane and carbon oxides to produce a cracked gas stream comprising the acetylene. Embodiment 6 is the process of embodiment 5, further comprising separating a gaseous C₂+ hydrocarbons stream from the cracked gas stream. Embodiment 7 is the process of embodiment 6, further comprising contacting the C₂+ hydrocarbons stream with an acetylene extraction solvent to produce: the liquid composition of step (a); and acetylene. Embodiment 8 is the process of embodiment 7, wherein the acetylene extraction solvent comprises the third liquid stream of step (c). Embodiment 9 is the process of any one of embodiments 1 to 8, further comprising cooling the third liquid stream to a temperature of 10° C. to 50° C. Embodiment 9 is the process of any one of embodiments 1 to 9, further comprising: separating in a regeneration column a portion of the third liquid stream of step (c) comprising the acetylene extraction solvent into a regenerated acetylene extraction solvent and a gaseous hydrocarbon stream comprising acetylene and light oligomers; providing the regenerated acetylene extraction solvent to step (b); and contacting in the scrubber column of step (b) the regenerated acetylene extraction solvent with the first gaseous hydrocarbon stream and the recovered ethylene stream from step (c). Embodiment 11 is the process of embodiment 10, further comprising cooling the regenerated acetylene extraction solvent prior to providing the regenerated acetylene extraction solvent to step (b). Embodiment 12 is the process of any one of embodiments 1 to 11, further comprising: flashing the first liquid stream of step (a) under reduced pressure, preferably 0.5 to 1.5 MPa(g) to produce: a vapor stream that comprises ethylene and acetylene; and a fourth liquid stream comprising the acetylene extraction solvent; and combining the vapor stream with first gaseous hydrocarbon stream. Embodiment 13 is the process of embodiment 12, wherein the combined stream is cooled to about 20 to 30° C. prior to being provided to step (c). Embodiment 14 is the process of any one of embodiments 11 to 13, wherein the fourth liquid stream is provided to the a lower section of the ethylene stripper unit of step (c). Embodiment 15 is the process of any one of embodiments 1 to 14, further comprising removing the recovered gaseous ethylene stream from an upper section of the ethylene stripper unit and compressing the crude gaseous ethylene stream prior to step (d). Embodiment 16 is the process of any one of embodiments 1 to 15, wherein hydrogenating step (a) comprises contacting the composition comprising acetylene and an acetylene extraction solvent with a hydrogenation catalyst, preferably a supported palladium and zinc catalyst. Embodiment 17 is the process of any one of embodiments 1 to 16, wherein the hydrogenating conditions comprise a temperature of 90° C. to 125° C. and a pressure of 1.5 MPa(g) to 1.8 MPa(g). Embodiment 18 is the process of any one of embodiments 1 to 17, further comprising preheating a hydrogenation feed mixture comprising a portion of the separated gaseous C₂+ hydrocarbons stream and the composition comprising acetylene and an acetylene extraction solvent to 90° C. to 125° C. prior to step (a). Embodiment 19 is the process of any one of embodiments 1 to 18, wherein the acetylene extraction solvent is an organic amine, acetone, methanol, paraffinic or olefinic liquids, preferably dimethylformamide, most preferably, N-methyl, 2-pyrrolidone. Embodiment 20 is the process of any one of embodiments 1 to 19, wherein the composition being hydrogenated in step (a) further comprises a sufficient amount of a cracked gas stream such that a H₂/acetylene ratio in the composition contacted in the hydrogenation unit of step (a) is 1.5:1 to 3:1.

The following includes definitions of various terms and phrases used throughout this specification.

The terms “about” or “approximately” are defined as being close to as understood by one of ordinary skill in the art. In one non-limiting embodiment, the terms are defined to be within 10%, preferably, within 5%, more preferably, within 1%, and most preferably, within 0.5%.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. With respect to the transitional phrase “consisting essentially of,” in one non-limiting aspect, a basic and novel characteristic of the processes of the present invention are their abilities to efficiently purify and recover polymer-grade ethylene from cracked gas streams.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding of the present invention, reference is now made to the following descriptions taken in conjunction with the accompanying drawing, in which:

FIG. 1 shows a prior art system for recovering ethylene; and

FIG. 2 shows a system for recovering and purifying ethylene, according to embodiments of the invention.

### DETAILED DESCRIPTION OF THE INVENTION

A discovery has been made of a process for recovering ethylene from cracked gas that includes acetylene scrubbing, by an acetylene scrubbing column, and ethylene stripping, in an ethylene distillation column, of streams that include ethylene. The process is such that the acetylene scrubbing and the ethylene stripping compensates for low conversion rates of acetylene to ethylene at a liquid phase hydrogenation step, in the ethylene recovery process, and still produce polymer-grade ethylene from the process.

Cracked gas that is rich in acetylene can be produced by thermal pyrolysis of hydrocarbons, one-stage partial oxidation of hydrocarbons, or two-stage combustion/pyrolysis of hydrocarbons. For example, the acetylene can be produced in the cracked gas by partial oxidation of methane, methane pyrolysis, or heating a composition of methane and carbon oxides. It may be particularly advantageous to use low cost natural gas, remote gas, or shale gas as the hydrocarbon feedstock. Partial oxidation and combustion/pyrolysis are typical methods used for cracking methane rich feedstocks. The cracked gas can be rich in hydrogen, carbon monoxide, carbon dioxide, and unconverted methane. The cracked gas can include acetylene in the range of 5 to 15 vol %. This acetylene can be converted to high value added ethylene by hydrogenation. According to embodiments of the invention, high ethylene yield is achievable by carrying out liquid phase acetylene hydrogenation using solvents such as N-methyl-2-pyrrolidone (NMP) or dimethyl formamide (DMF). In embodiments of the invention, acetylene scrubbing and ethylene stripping columns are integrated into the reaction system enabling near 100% ethylene recovery and acetylene product that meets specifications downstream without the need of vapor phase acetylene converter/polishing reactors.

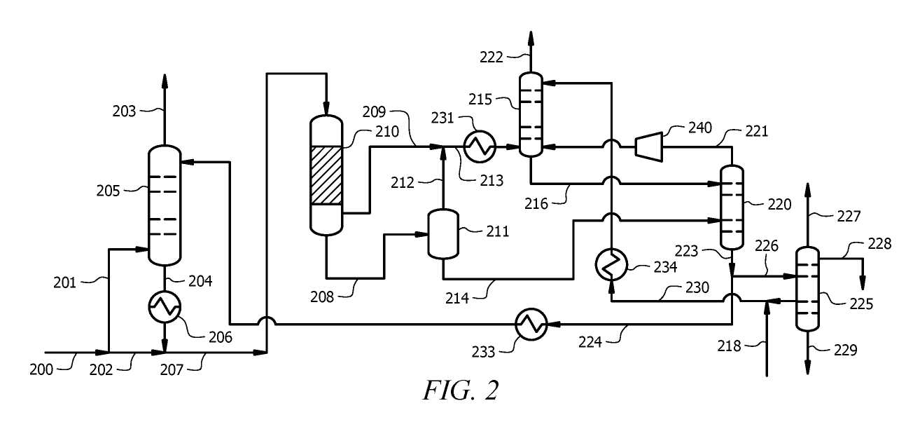

Referring to FIG. 2, a system of the present invention for purifying and recovering ethylene from a cracked gas stream that involves acetylene hydrogenation and removal from cracked gas, according to embodiments of the invention, is shown. The feedstock for the system can be a crude cracked gas stream from a reactor that is compressed to 280 to 300 psid (1.93 to 2.07 MPa(g)) and fed to an amine unit (not shown). The amine unit removes acid gases, mostly CO₂, H₂S, and/or COS to form cracked gas stream 200. Cracked gas stream 200 can include gaseous C₂+ hydrocarbons. Cracked gas stream 200 can be split into two fractions. The mass flow rate of the first fraction, stream 201, can be approximately ¾ of the mass flow rate of cracked gas stream 200. As shown, stream 201 can be fed to the bottom of acetylene absorber column 205 where it contacts acetylene extraction solvent stream 224 entering the top of acetylene absorber column 205. In some embodiments, stream 201 enters the top of acetylene absorber column 205 and extraction solvent stream 224 enter the bottom of the acetylene absorber column. Acetylene extraction solvent stream 224 can include one of organic amine, acetone, methanol, paraffinic or olefinic liquids, preferably dimethylformamide, most preferably, N-methyl, 2-pyrrolidone, or combinations thereof. The mass flow rate of the second fraction, stream 202, can be approximately ¼ of the mass flow rate of cracked gas stream 200. Stream 202 can bypass acetylene absorber column 205 and be fed directly into hydrogenation reactor 210. In this way, stream 202 acts as a source of hydrogen for hydrogenation reactor 210. The split of cracked gas stream 200 may be adjusted so that H₂/acetylene ratio fed into the hydrogenation reactor is in the range of 1.5:1 to 3:1.

Acetylene absorber column 205 can be a packed column that has sufficient theoretic stages, typically 14, to achieve over 99% acetylene recovery. Acetylene absorber column 205 can be designed to have sufficient theoretic stages that can recover substantial amounts of ethylene and acetylene from cracked gas stream 200, particularly when cracked gas stream 200 is rich in ethane, propane, or butane. Acetylene extraction solvent stream 224, preferably N-methyl-2-pyrrolidone (NMP), can be fed in a countercurrent direction to stream 201 (e.g., overhead) to acetylene absorber column 205, where acetylene extraction solvent stream 224 preferentially absorbs acetylene into the liquid phase because of acetylene's much higher solubility in acetylene extraction solvent stream 224 than ethylene, methane, CO, and H₂.

Absorber overhead vent gas 203 may be sent to the combustor and used as fuel gas. Absorber bottom solvent stream 204, which is acetylene rich, flowing from the bottom of acetylene absorber column 205, can be preheated using preheater 206 to a temperature in the range 200 to 250° F. (93.3 to 121° C.). Subsequently, absorber bottom solvent stream 204 can be combined with stream 202 to form mixed stream 207, which is a 2-phase flow where both phases are fed concurrently into hydrogenation reactor 210. As shown, stream 202 and mixed stream 207 enter hydrogenation reactor 210 as one stream. In some embodiments, both streams enter the hydrogenation reactor as independent streams.

Hydrogenation reactor 210 can run at trickle mode and be packed with a hydrogenation catalyst for liquid phase selective acetylene hydrogenation. A variety of hydrogenation catalysts can be used in the context of the present invention. In one embodiment, the hydrogenation catalyst can be a palladium (Pd)/zinc (Zn) on alumina catalyst (e.g., 1/16^{th} inch spheres of palladium (Pd)/zinc (Zn) on alumina catalysts). The weight hourly space velocity (WHSV_{C2H2}) can be at 0.05 to 0.2 hr⁻¹ (lb C₂H₂/lb cat/hr) on the basis of acetylene in the feed (mixed stream 207). The feed rate of acetylene extraction solvent stream 224 to acetylene absorber column 205 can be set such that the acetylene concentration in the liquid is no more than 1.5 wt. % and that the adiabatic temperature rise across the reactor is less than 60° F. (33.3° C.). The hydrogenation reactor inlet temperature and pressure are typically at 200° F. (93.3° C.) and 250 psig (1.72 MPa(g)), respectively. However, other temperatures and pressures can be used in the context of the present invention. The outlet temperature and pressure drop are typically at 230 to 260° F. (110 to 126.6° C.) and 15 to 40 psig (0.10 to 0.28 MPa), respectively. However, other temperatures and pressure can be used in the context of the present invention. In embodiments of the invention, the hydrogenating conditions in hydrogenation reactor 210 can include a temperature of 90° C. to 125° C. and a pressure of 1.5 MPa(g) to 1.8 MPa(g), and all ranges and values there between including 90 to 95° C., or 95 to 100° C., or 100 to 105° C., or 105 to 110° C., or 110 to 115° C., 115 to 120° C., or 120 to 125° C., and 1.5 MPa(g), 1.6 MPa(g), 1.7 MPa(g) or 1.8 MPa(g). In embodiments of the invention, the hydrogenating conditions in hydrogenation reactor 210 includes a temperature in the range 90 to 95° C., or 95 to 100° C., or 100 to 105° C., or 105 to 110° C., or 110 to 115° C., 115 to 120° C., or 120 to 125° C., and all ranges and values there between. In hydrogenation reactor 210, hydrogenation of a composition that includes acetylene and an acetylene extraction solvent occurs under conditions sufficient to produce hydrogenation reactor effluent vapor stream 209 and hydrogenation reactor liquid stream 208. In embodiments of the invention, hydrogenation reactor effluent vapor stream 209 includes ethylene and acetylene and hydrogenation reactor liquid stream 208 includes ethylene and acetylene gases dissolved in acetylene extraction solvent. In embodiments of the invention, hydrogenation reactor liquid stream 208 can include more acetylene than hydrogenation reactor effluent vapor stream 209. The acetylene conversion by hydrogenation reactor 210 may be in the range of 93 to 99.9% with selectivity near 97%.

At the bottom effluent reservoir of hydrogenation reactor 210, hydrogenation reactor effluent vapor stream 209 and hydrogen reactor liquid stream 208 disengages. Hydrogen reactor liquid stream 208 can be depressurized to 80 to 200 psig (0.55 MPa(g) to 1.38 MPa(g)) in flash tank 211, which produces vapor stream 212. Vapor stream 212 includes recovered ethylene. Vapor stream 212 mixes with hydrogenation reactor effluent vapor stream 209 to form mixed hot vapor stream 213. Mixed hot vapor stream 213 can be cooled by air-fan heat exchanger 231 to near ambient temperature and can then be routed to a lower section of acetylene scrubbing column 215. Acetylene scrubbing column 215 can have stages sufficient to remove acetylene to 0.1 ppm or less in crude ethylene product stream 222. In embodiments of the invention, mixed hot vapor stream 213 is cooled to a temperature in the range 20 to 30° C., and all ranges and values there between including 20° C., 21° C., 22° C., 23° C., 24° C., 25° C., 26° C., 27° C., 28° C., 29° C., or 30° C. In embodiments of the invention, acetylene scrubbing column 215 can have approximately 20 to 30 theoretic stages, preferably 22 to 26 stages. Regenerated solvent stream 230 can be chilled by chiller 234 and fed to the top of acetylene scrubbing column 215. In embodiments of the invention, acetylene scrubbing column 215 reduces the acetylene content of crude ethylene product stream 222 to 0.1 ppm or less.

Regenerated solvent stream 230, in embodiments of the invention, can be chilled to a temperature in the range 10° C. to 50° C., and all ranges and values there between including 10° C. to 20° C., 20° C. to 30° C., 30° C. to 40° C., or 40° C. to 50° C. In embodiments of the invention, acetylene scrubbing column 215 can be operated at a pressure of 5 to 75 psig (0.03 to 0.52 MPa(g)), and all ranges and values there between including 0.03 to 0.52 MPa(g), or 0.03 to 0.05 MPa(g), or 0.05 to 0.1 MPa(g), or 0.15 to 0.2 MPa(g), or 0.2 to 0.25 MPa(g), or 0.25 to 0.3 MPa(g), or 0.35 to 0.4 MPa(g), or 0.45 to 0.52 MPa(g). In embodiments of the invention, acetylene scrubbing column 215 enhances the level of recovery of ethylene from hydrogenation reactor effluent. According to embodiments of the invention, in acetylene scrubbing column 215, hydrogenation reactor effluent vapor stream 209 is contacted with regenerated solvent stream 230 (an acetylene extraction solvent) under contacting conditions sufficient to extract a portion of the acetylene from hydrogenation reactor effluent vapor stream 209 to produce acetylene scrubbing column bottom stream 216 (a liquid stream including ethylene and acetylene gases dissolved in acetylene extraction solvent) and crude ethylene product stream 222.

In embodiments of the invention, gaseous crude ethylene product stream 222 includes 1 ppm or less of acetylene, and/or the ethylene recovery efficiency is at least 80%, preferably 100%. In embodiments of the invention, gaseous crude ethylene product stream 222 can include 0 ppm to 1 ppm acetylene, and all ranges and values there between including 0 ppm, 0.1 ppm, 0.2 ppm, 0.3 ppm, 0.4 ppm, 0.5 ppm, 0.6 ppm, 0.7 ppm, 0.8 ppm, 0.9 ppm, or 1 ppm acetylene. In embodiments of the invention, the ethylene recovery efficiency is in a range of 80% to 100%, and all ranges and values there between. In embodiments of the invention, acetylene scrubbing column 215 reduces the acetylene content of crude ethylene product stream 222 to 0.1 ppm or less. In embodiments of the invention, crude ethylene product stream 222 can be compressed.

In embodiments of the invention, acetylene scrubbing column bottom stream 216 is fed to the top of ethylene stripping distillation column 220, which can be operated at a lower pressure than acetylene scrubbing column 215. In embodiments of the invention, ethylene stripping distillation column 220 is operated in a pressure range of 5 to 70 psig (0.03 to 0.48 MPa(g)), and all ranges and values there between including 0.03 to 0.05 MPa(g), or 0.05 to 0.1 MPa(g), or 0.15 to 0.2 MPa(g), or 0.2 to 0.25 MPa(g), or 0.25 to 0.3 MPa(g), or 0.35 to 0.4 MPa(g), or 0.45 to 0.48 MPa(g).

Embodiments of the invention include contacting the hydrogenation reactor liquid stream (e.g., stream 208) with acetylene scrubbing column bottom stream 216 in ethylene stripping distillation column 220 under separating conditions sufficient to produce overhead vapor stream 221 (which includes recovered gaseous ethylene) and ethylene stripping column bottom stream 223 (which is a liquid stream that includes acetylene dissolved in the acetylene extraction solvent). In embodiments of the invention, ethylene stripping distillation column 220 can have approximately 10 to 20 theoretic stages, preferably with 12 to 16 theoretic stages. In embodiments of the invention, ethylene stripping distillation column 220 can have no overhead condenser, but can be equipped with a reboiler at the bottom. Ethylene stripping distillation column 220 is configured to strip dissolved ethylene from streams sent to it, and it can do so with reduced amount of acetylene in overhead streams, for enhanced ethylene recovery in those overhead streams.

In embodiments of the invention, prior to contacting hydrogenation reactor liquid stream 208 with acetylene scrubbing column bottom stream 216 in ethylene stripping distillation column 220, hydrogenation reactor liquid stream 208 can be depressurized in flash tank 211 to form hot liquid stream 214 flowing from the bottom of flash tank 211 and vapor stream 212 (which can include ethylene and acetylene). Vapor stream 212 can be combined with hydrogenation reactor effluent vapor stream 209 to form a mixed vapor stream 213 before being fed to acetylene scrubbing column 215. Flash tank 211 can be depressurized to a pressure in the range 0.5 to 1.5 MPa(g) to form hot liquid stream 214 and vapor stream 212. In embodiments of the invention, flash tank 211 can be depressurized to a pressure in the range 0.5 to 1.5 MPa(g), and all ranges and values there between including 0.5 MPa(g), 0.6 MPa(g), 0.7 MPa(g), 0.8 MPa(g), 0.9 MPa(g), 1.0 MPa(g), 1.1 MPa(g), 1.2 MPa(g), 1.3 MPa(g), 1.4 MPa(g), or 1.5 MPa(g).

Hot liquid stream 214 can be fed to the mid-upper section of ethylene stripping distillation column 220. Overhead vapor stream 221 can be compressed by compressor 240 and routed to the bottom of acetylene scrubbing column 215 for ethylene recovery. Compressor 240 may be sized to overcome a pressure difference of 5 to 60 psid (0.3 MPa to 0.42 MPa). In embodiments of the invention, the operating pressure difference between acetylene scrubbing column 215 and ethylene stripping distillation column 220 allows efficient ethylene recovery and separation of residual acetylene dissolved in the solvent. In embodiments of the invention, the pressure in ethylene stripping column 220 is less than the pressure in acetylene scrubbing column 215. For example, in embodiments of the invention, acetylene scrubbing column 215 pressure can be from 0.01 MPa(g) to 0.5 MPa(g), preferably 0.03 MPa(g) to 0.5 MPa(g), and all ranges and values there between including 0.01 to 0.10 MPa(g), or 0.10 to 0.20 MPa(g), or 0.20 to 0.30 MPa(g), or 0.30 to 0.40 MPa(g), or 0.40 to 0.50 MPa(g). In embodiments of the invention, ethylene stripping distillation column 220 pressure may be 0.01 MPa(g) to 0.4 MPa(g) less than acetylene scrubbing column 215 pressure. Ethylene stripping column bottom stream 223 may be split into two fractions (acetylene extraction solvent stream 224 and stream 226).

In embodiments of the invention, acetylene extraction solvent stream 224 can include about 70 to 75% of ethylene stripping column bottom stream 223. Acetylene extraction solvent stream 224 can be chilled by chiller 233 to a temperature in the range of 10° C. to 50° C. and then used as acetylene extraction solvent for acetylene absorber column 205. In embodiments of the invention, about 25 to 30% of ethylene stripping column bottom stream 223 can include stream 226. Stream 226 may be routed to solvent regeneration column 225, where light hydrocarbons are purged as overhead vapor stream 227 and overhead condensed liquid stream 228. Vapor stream 227 may include acetylene and light oligomers. Heavy hydrocarbons are purged through regeneration column bottom stream 229.

In embodiments of the invention, regenerated solvent stream 230, from solvent regeneration column 225, can be free of acetylene and low in light hydrocarbons, such as C₄ hydrocarbons, and heavy hydrocarbons, such as green oils. Regenerated solvent stream 230 can be chilled by chiller 234 and recycled to acetylene scrubbing column 215 as clean solvent for acetylene scrubbing operation, where regenerated solvent stream 230 contacts mixed hydrogenation reactor effluent vapor stream 213 and overhead vapor stream 221. In embodiments of the invention, solvent regeneration column 225 can have 10 to 20 theoretic stages, preferably with 12 to 18 stages, and may be operated under a vacuum of 50-600 torr to minimize bottoms temperature. A small N-methyl-2-pyrrolidone solvent makeup stream 218 can be added to regenerated solvent recycle stream 230 to compensate the light hydrocarbons and heavy hydrocarbons purge.

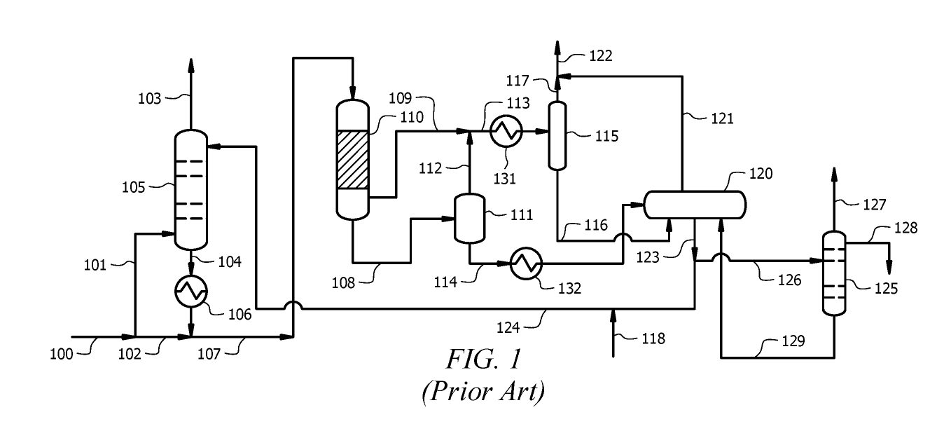

Below, described are simulations of the prior art system of FIG. 1 that have been run (Examples 1 and 2) for comparison with a simulation of the system of the current invention (e.g., FIG. 2), according to embodiments of the invention (Example 3). Comparing Examples 1 and 2 with Example 3 shows that Example 3 is an improvement over Examples 1 and 2. In Example 4, sensitivity calculations were performed on Examples 1, 2, and 3.

### EXAMPLES

### Example 1 (Comparative)

In the simulation of the operation of the prior art system, pipeline natural gas was cracked in a pilot two-stage combustor/pyrolysis reactor. The cracked gas was compressed and treated in an amine unit to remove most of carbon dioxide (CO₂) in the cracked gas. This sweetened cracked gas formed cracked gas stream 100, which serves as feed to the prior art system as shown in FIG. 1. The prior art system is configured in the simulation to convert acetylene into ethylene by acetylene hydrogenation followed by flash separation steps. The composition, flow rate and properties of process streams of the prior art system, as simulated, are presented in Table 1.

Referring to FIG. 1, cracked gas stream 100, flowing at a rate of 120 lb/hr was split into stream 101 (96 lb/hr) and stream 102 (24 lb/hr). Stream 101 was fed to acetylene absorber column 105, and stream 102 bypasses acetylene absorber column 105. Acetylene extraction solvent stream 124 flowing at a rate of 1500 lb/hr was fed to the top of acetylene absorber column 105 (a 14-stage acetylene absorber). Absorber bottom solvent stream 104 was preheated by preheater 106 and mixed with stream 102 (bypass cracked gas) and fed to acetylene hydrogenation reactor 110. Mixed stream 107 contained 11.4 lb/hr acetylene and flowed at a rate of 1529 lb/hr. Hydrogenation reactor 110 had an internal diameter of 5 inches and a height of 15 feet of catalysts packing and was running at an acetylene based weight hourly space velocity (WHSV_{C2H2}) of 0.104 lb C₂H₂/lb cat/hr.

Acetylene hydrogenation reactor 110, operated at 250 psig inlet pressure with 17 psid pressure drop across the reactor, had a temperature increase from 188° F. (86.6° C.) at the inlet to 230° F. (110° C.) at the outlet at 97% acetylene conversion rate. Hydrogenation reactor liquid stream 108, flow of 1502 lb/hr, was depressurized to 150 psig (1.03 MPa(g)) in flash tank 111. Flashed vapor stream 112 was combined with reactor effluent vapor stream 109. Mixed hot vapor stream 113 was cooled and depressurized to 75 psig (0.517 MPa(g)) in flash vessel 115. Flashed vapor stream 117 was combined with vapor stream 121 from solvent accumulation tank 120. Flow of crude ethylene product stream was 29 lb/hr and included 7.73 lb/hr ethylene and 0.06 lb/hr acetylene. Crude ethylene product stream 122 was calculated in the simulation to have 8064 ppm acetylene at 100% ethylene basis with an ethylene recovery efficiency at 65.3%.

Knock-out liquid stream 114, from flash tank 111, was cooled and depressurized to 75 psig (0.517 MPa(g)) in the solvent accumulation vessel 120. A small flow of 38 lb/hr withdrawn from solvent accumulation vessel 120 (stream 126) was sent for light hydrocarbon removal and regeneration. A large fraction of flow from solvent accumulation vessel 120 (stream 123) was combined with makeup fresh solvent stream 118 of 3.8 lb/hr to form acetylene extraction solvent stream 124 for solvent recycling at a flow rate of 1500 lb/hr. Acetylene extraction solvent stream 124 contained 0.28 lb/hr acetylene and 4 lb/hr ethylene, in the simulation.

### Example 2 (Comparative)

Similar to Example 1, cracked gas stream 100, was used as feed to the prior art system shown as shown in FIG. 1. The system was used to convert acetylene into ethylene via acetylene hydrogenation followed by flash separation steps. The difference between Example 1 and Example 2 was that in Example 2, both flash vessel 111 and flash vessel 115 were operated at 5 psig (0.03 MPa(g)) instead of 75 psig (0.517 MPa(g)). At this pressure in the flash vessels, based on the process mass balances presented in Table 2, crude ethylene product stream 122 was at 32.3 lb/hr with 9.76 lb/hr ethylene and 0.07 lb/hr acetylene. This crude ethylene product stream was calculated to have 7955 ppm acetylene at 100% ethylene basis with ethylene recovery efficiency at 82.5%.

### Example 3 (Process of the Current Invention)

Referring to FIG. 2, in a simulation of the operation of the system of the current invention, pipeline natural gas was cracked in a pilot two-stage combustor/pyrolysis reactor. The cracked gas was compressed and treated in an amine unit to have most of its carbon dioxide (CO₂) removed. This sweetened cracked gas formed cracked gas stream 200, which serves as feed for the system. The system of the current invention was configured in the simulation to convert acetylene into ethylene via acetylene hydrogenation with acetylene scrubbing and ethylene recovery operations. The composition, flow rates and properties of process streams of the system of the current invention, as simulated, are presented in Table 3.

In the simulation, cracked gas stream 200, flowed at a rate of 120 lb/hr, was split into stream 201 (96 lb/hr) and stream 202 (24 lb/hr). Stream 201 was fed to acetylene absorber column 205, and stream 202 bypassed acetylene absorber column 205. Acetylene extraction solvent stream 224 flowed at a rate of 1500 lb/hr was fed to the top of acetylene absorber column 205 (a 14-stage acetylene absorber). Absorber bottom solvent stream 204 was preheated by preheater 206 and mixed with stream 202 (bypass cracked gas) and fed to hydrogenation reactor 210. Mixed stream 207 contained 11.4 lb/hr acetylene and flowed at a rate of 1529 lb/hr. Hydrogenation reactor 210 had an internal diameter of 5 inches and a height of 15 feet of catalysts packing and was run at an acetylene based weight hourly space velocity (WHSV_{C2H2}) of 0.104 lb C₂H₂/lb cat/hr.

Hydrogenation reactor 210, operated at 250 psig inlet pressure with 17 psid pressure drop across the reactor, had a temperature increase from 188° F. (86.6° C.) at the inlet to 230° F. (110° C.) at the outlet, at 97% acetylene conversion rate. Reactor liquid effluent 208 with a flow of 1502 lb/hr was depressurized to 150 psig (1.03 MPa(g)) in flash tank 211. Flashed vapor stream 212 was combined with hydrogenation reactor effluent vapor stream 209. Mixed hot vapor stream 213 was fed to the bottom of acetylene scrubbing column 215 (a 24-stage acetylene scrubbing column), which was operated at an overhead pressure of 35 psig. Regenerated solvent stream 230 was chilled to 85° F. (29.4° C.) and fed at a rate of 560 lb/hr to the top of acetylene scrubbing column 215.

Crude ethylene product stream 222 was withdrawn at a rate of 35.1 lb/hr from acetylene scrubbing column 215 for further downstream separation. Crude ethylene product stream 222 contained 11.85 lb/hr ethylene and 0.000013 lb/hr acetylene. Crude ethylene product stream 222 was calculated to have 1.2 ppm acetylene at 100% ethylene basis with a 100% ethylene recovery efficiency. Acetylene scrubbing column bottom stream 216 was fed to the top of ethylene stripping distillation column 220, which had 14 theoretic stages and operated at 15 psig (0.10 MPa(g)) head pressure. Hot liquid stream 214, from flash tank 211, was fed to ethylene stripping distillation column 220 at the 4^{th} tray from the top. Stream 226, a portion of the hot liquid from ethylene stripping column bottom stream 223, was sent to solvent regeneration column 225 at a rate of 561 lb/hr for light and heavy hydrocarbon purge. The rest of the ethylene stripper bottoms, acetylene extraction solvent stream 224, was chilled to 85° F. (29.4° C.) and fed back into the acetylene absorber column 205 at a rate of 1500 lb/hr.

Solvent regeneration was carried out in solvent regeneration column 225 (a 12 theoretic-stage distillation column), where dissolved residual acetylene was stripped to form vapor stream 227 at a rate of 0.1 lb/hr and condensable light hydrocarbons, such as C₄s, are purged overhead at a rate of 5.5 lb/hr through overhead condensed liquid stream 228. Heavy hydrocarbons, such as green oils, were purged through regeneration column bottom stream 229 at a rate of 5.3 lb/hr. A liquid side-draw taken from the 8^{th} tray at 550 lb/hr from the top was combined with a make-up solvent feed 218 at 10.6 lb/hr and chilled to 85° F. (29.4° C.) before it is used as clean regenerated solvent stream 230, which was routed to acetylene scrubbing column 215. A large fraction of the ethylene stripper bottoms, acetylene extraction solvent stream 224, was chilled to 85° F. (29.4° C.) and recycled back to acetylene absorber column 205 at a circulation rate of 1500 lb/hr. Acetylene extraction solvent stream 224 contained 0.25 lb/hr acetylene and a negligible amount of ethylene, in the simulation. Thus, Example 3 was an improvement over Examples 1 and 2.

### Example 4 (Sensitivity of Examples 1-3)

Sensitivity calculations were performed on Examples 1, 2, and 3 with acetylene fractional conversions ranging from 0.93 to 0.999. The results for the sensitivity calculations are provided in Table 4. From the data in Table 4 the system of the current invention, which had an acetylene scrubbing column and an ethylene stripping column, according to embodiments of the invention, the quality of ethylene product was not affected by the variations of acetylene conversion at the liquid phase hydrogenation step. Thus, even at an acetylene fractional conversion as low as 93%, the product ethylene produced, according to embodiments of the invention, comfortably met a polymer grade specification of less than 5 ppm acetylene or less than 2 ppm acetylene, or 2 to 5 ppm acetylene. On the other hand, the prior art system that had flash separation steps, required an acetylene converter and polishing reactors downstream for product purification. For the prior art system, if acetylene fractional conversion fell below 0.97 and acetylene concentration exceeded 1% in ethylene, multiple-bed acetylene converters would become necessary.

TABLE 1

| Stream (Example 1) | 100 | 101 | 102 | 103 | 104 | 107 | 109 | 108 |
|---|---|---|---|---|---|---|---|---|
| Mole Flow lbmol/hr | | | | | | | | |
| HYDROGEN | 3.830185 | 3.064148 | 0.766037 | 3.053191 | 0.01324 | 0.779277 | 0.326068 | 0.017965 |
| NITROGEN | 0.078785 | 0.063028 | 0.015757 | 0.062846 | 2.98E−04 | 0.016055 | 0.01523 | 8.25E−04 |
| OXYGEN | 0.016163 | 0.01293 | 3.23E−03 | 0.010617 | 2.78E−03 | 6.01E−03 | 5.26E−03 | 7.54E−04 |
| CARBON- | 2.703926 | 2.163141 | 0.540785 | 2.158183 | 0.019737 | 0.560522 | 0.507862 | 0.05266 |
| MONOXIDE | | | | | | | | |
| CARBON- | 0.100633 | 0.080506 | 0.020127 | 0.076253 | 0.021349 | 0.041475 | 0.022074 | 0.019402 |
| DIOXIDE | | | | | | | | |
| METHANE | 1.043907 | 0.835126 | 0.208782 | 0.832957 | 0.020258 | 0.22904 | 0.193434 | 0.035606 |
| ACETYLENE | 0.430527 | 0.344421 | 0.086105 | 4.30E−03 | 0.350715 | 0.436821 | 2.06E−03 | 0.011023 |
| ETHYLENE | 0.035769 | 0.028615 | 7.15E−03 | 0.167354 | 3.96E−03 | 0.011117 | 0.251075 | 0.170653 |
| ETHANE | 0 | 0 | 0 | 1.84E−03 | 1.04E−17 | 1.04E−17 | 4.66E−03 | 2.33E−03 |
| PROPADIENE | 5.17E−03 | 4.14E−03 | 1.03E−03 | 2.03E−04 | 4.25E−03 | 5.29E−03 | 8.74E−05 | 3.36E−04 |
| METHYL- | 4.25E−05 | 3.40E−05 | 8.50E−06 | 2.16E−06 | 3.41E−05 | 4.26E−05 | 9.93E−07 | 2.41E−06 |
| ACETYLENE | | | | | | | | |
| PROPYLENE | 0 | 0 | 0 | 2.20E−03 | 8.53E−10 | 8.53E−10 | 2.35E−03 | 2.45E−03 |
| PROPANE | 0 | 0 | 0 | 3.58E−05 | 1.14E−14 | 1.14E−14 | 6.47E−05 | 4.19E−05 |
| 1,3-BUTADIENE | 0 | 0 | 0 | 1.56E−03 | 3.22E−03 | 3.22E−03 | 8.74E−04 | 4.96E−03 |
| ISOBUTYLENE | 0 | 0 | 0 | 1.25E−04 | 4.18E−06 | 4.18E−06 | 6.37E−05 | 1.37E−04 |
| CIS-2-BUTENE | 0 | 0 | 0 | 9.78E−05 | 1.08E−06 | 1.08E−06 | 4.85E−05 | 1.05E−04 |
| TRANS-2-BUTENE | 0 | 0 | 0 | 6.07E−05 | 1.54E−05 | 1.54E−05 | 1.04E−05 | 7.92E−05 |
| 1-HEXENE | 2.59E−03 | 2.07E−03 | 5.19E−04 | 2.33E−04 | 3.74E−03 | 4.26E−03 | 1.70E−04 | 1.96E−03 |
| N-HEXANE | 0 | 0 | 0 | 1.20E−03 | 2.48E−03 | 2.48E−03 | 7.78E−04 | 3.83E−03 |
| 1-HEXADECENE | 0 | 0 | 0 | 7.62E−08 | 4.20E−03 | 4.20E−03 | 8.17E−07 | 4.21E−03 |
| N-METHYL-2- | 0 | 0 | 0 | 1.06E−03 | 15.0533 | 15.0533 | 5.32E−03 | 15.04799 |
| PYRROLIDONE | | | | | | | | |
| Mole Frac | | | | | | | | |
| HYDROGEN | 0.464394 | 0.464394 | 0.464394 | 0.478983 | 8.54E−04 | 0.045431 | 0.243792 | 1.17E−03 |
| NITROGEN | 9.55E−03 | 9.55E−03 | 9.55E−03 | 9.86E−03 | 1.92E−05 | 9.36E−04 | 0.011387 | 5.36E−05 |
| OXYGEN | 1.96E−03 | 1.96E−03 | 1.96E−03 | 1.67E−03 | 1.79E−04 | 3.50E−04 | 3.93E−03 | 4.90E−05 |
| CARBON- | 0.32784 | 0.32784 | 0.32784 | 0.338575 | 1.27E−03 | 0.032678 | 0.379714 | 3.42E−03 |
| MONOXIDE | | | | | | | | |
| CARBON- | 0.012201 | 0.012201 | 0.012201 | 0.011962 | 1.38E−03 | 2.42E−03 | 0.016504 | 1.26E−03 |
| DIOXIDE | | | | | | | | |
| METHANE | 0.12657 | 0.12657 | 0.12657 | 0.130674 | 1.31E−03 | 0.013353 | 0.144625 | 2.32E−03 |
| ACETYLENE | 0.0522 | 0.0522 | 0.0522 | 6.75E−04 | 0.022622 | 0.025466 | 1.54E−03 | 7.17E−04 |
| ETHYLENE | 4.34E−03 | 4.34E−03 | 4.34E−03 | 0.026254 | 2.56E−04 | 6.48E−04 | 0.187722 | 0.011098 |
| ETHANE | 0 | 0 | 0 | 2.88E−04 | 6.70E−19 | 6.05E−19 | 3.48E−03 | 1.52E−04 |
| PROPADIENE | 6.27E−04 | 6.27E−04 | 6.27E−04 | 3.19E−05 | 2.74E−04 | 3.08E−04 | 6.54E−05 | 2.18E−05 |
| METHYL- | 5.15E−06 | 5.15E−06 | 5.15E−06 | 3.39E−07 | 2.20E−06 | 2.48E−06 | 7.43E−07 | 1.57E−07 |
| ACETYLENE | | | | | | | | |
| PROPYLENE | 0 | 0 | 0 | 3.45E−04 | 5.50E−11 | 4.97E−11 | 1.76E−03 | 1.59E−04 |
| PROPANE | 0 | 0 | 0 | 5.61E−06 | 7.37E−16 | 6.66E−16 | 4.84E−05 | 2.72E−06 |
| 1,3-BUTADIENE | 0 | 0 | 0 | 2.45E−04 | 2.07E−04 | 1.87E−04 | 6.53E−04 | 3.23E−04 |
| ISOBUTYLENE | 0 | 0 | 0 | 1.95E−05 | 2.69E−07 | 2.43E−07 | 4.76E−05 | 8.91E−06 |
| CIS-2-BUTENE | 0 | 0 | 0 | 1.53E−05 | 6.98E−08 | 6.31E−08 | 3.63E−05 | 6.86E−06 |
| TRANS-2-BUTENE | 0 | 0 | 0 | 9.52E−06 | 9.90E−07 | 8.95E−07 | 7.79E−06 | 5.15E−06 |
| 1-HEXENE | 3.14E−04 | 3.14E−04 | 3.14E−04 | 3.65E−05 | 2.41E−04 | 2.48E−04 | 1.27E−04 | 1.28E−04 |
| N-HEXANE | 0 | 0 | 0 | 1.89E−04 | 1.60E−04 | 1.44E−04 | 5.82E−04 | 2.49E−04 |
| 1-HEXADECENE | 0 | 0 | 0 | 1.20E−08 | 2.71E−04 | 2.45E−04 | 6.11E−07 | 2.73E−04 |
| N-METHYL-2- | 0 | 0 | 0 | 1.66E−04 | 0.970956 | 0.877584 | 3.97E−03 | 0.978584 |
| PYRROLIDONE | | | | | | | | |

| Stream (Example 1) | 114 | 112 | 113 | 117 | 116 | 121 | 122 |
|---|---|---|---|---|---|---|---|
| Mole Flow lbmol/hr | | | | | | | |
| HYDROGEN | 8.43E−03 | 9.53E−03 | 0.335598 | 0.335597 | 1.43E−06 | 6.09E−03 | 0.341692 |
| NITROGEN | 3.83E−04 | 4.42E−04 | 0.015672 | 0.015671 | 7.85E−08 | 2.65E−04 | 0.015936 |
| OXYGEN | 4.96E−04 | 2.58E−04 | 5.51E−03 | 5.51E−03 | 2.15E−06 | 2.27E−05 | 5.53E−03 |
| CARBON- | 0.032919 | 0.019741 | 0.527603 | 0.527598 | 5.09E−06 | 0.017766 | 0.545364 |
| MONOXIDE | | | | | | | |
| CARBON- | 0.018126 | 1.28E−03 | 0.023349 | 0.023341 | 8.85E−06 | 6.02E−04 | 0.023942 |
| DIOXIDE | | | | | | | |
| METHANE | 0.026626 | 8.98E−03 | 0.202414 | 0.202409 | 5.41E−06 | 8.08E−03 | 0.210486 |
| ACETYLENE | 0.010897 | 1.26E−04 | 2.19E−03 | 2.18E−03 | 8.59E−06 | 3.84E−05 | 2.22E−03 |
| ETHYLENE | 0.156482 | 0.014172 | 0.265247 | 0.265199 | 4.76E−05 | 0.010169 | 0.275368 |
| ETHANE | 2.08E−03 | 2.56E−04 | 4.91E−03 | 4.91E−03 | 6.02E−07 | 1.91E−04 | 5.10E−03 |
| PROPADIENE | 3.30E−04 | 5.28E−06 | 9.27E−05 | 9.25E−05 | 2.10E−07 | 1.89E−06 | 9.44E−05 |
| METHYL- | 2.36E−06 | 5.91E−08 | 1.05E−06 | 1.05E−06 | 2.04E−09 | 1.66E−08 | 1.07E−06 |
| ACETYLENE | | | | | | | |
| PROPYLENE | 2.31E−03 | 1.36E−04 | 2.49E−03 | 2.48E−03 | 1.28E−06 | 5.77E−05 | 2.54E−03 |
| PROPANE | 3.82E−05 | 3.62E−06 | 6.84E−05 | 6.83E−05 | 2.12E−08 | 1.58E−06 | 6.99E−05 |
| 1,3-BUTADIENE | 4.91E−03 | 5.26E−05 | 9.27E−04 | 9.22E−04 | 4.35E−06 | 1.40E−05 | 9.36E−04 |
| ISOBUTYLENE | 1.33E−04 | 3.78E−06 | 6.74E−05 | 6.74E−05 | 8.96E−08 | 1.31E−06 | 6.87E−05 |
| CIS-2-BUTENE | 1.03E−04 | 2.94E−06 | 5.15E−05 | 5.14E−05 | 6.16E−08 | 1.12E−06 | 5.25E−05 |
| TRANS-2-BUTENE | 7.86E−05 | 6.33E−07 | 1.11E−05 | 1.10E−05 | 1.64E−08 | 6.22E−07 | 1.17E−05 |
| 1-HEXENE | 1.95E−03 | 1.03E−05 | 1.81E−04 | 1.78E−04 | 2.40E−06 | 2.03E−06 | 1.80E−04 |
| N-HEXANE | 3.78E−03 | 4.76E−05 | 8.26E−04 | 8.22E−04 | 4.09E−06 | 1.07E−05 | 8.32E−04 |
| 1-HEXADECENE | 4.21E−03 | 5.27E−08 | 8.70E−07 | 5.77E−09 | 8.64E−07 | 5.91E−10 | 6.36E−09 |
| N-METHYL-2- | 15.04767 | 3.17E−04 | 5.63E−03 | 1.57E−04 | 5.48E−03 | 7.64E−06 | 1.64E−04 |
| PYRROLIDONE | | | | | | | |
| Mole Frac | | | | | | | |
| HYDROGEN | 5.51E−04 | 0.172156 | 0.240945 | 0.241911 | 2.56E−04 | 0.140671 | 0.238845 |
| NITROGEN | 2.50E−05 | 7.98E−03 | 0.011251 | 0.011297 | 1.41E−05 | 6.11E−03 | 0.01114 |
| OXYGEN | 3.24E−05 | 4.66E−03 | 3.96E−03 | 3.97E−03 | 3.86E−04 | 5.25E−04 | 3.87E−03 |
| CARBON- | 2.15E−03 | 0.356584 | 0.378795 | 0.380312 | 9.13E−04 | 0.410079 | 0.381213 |
| MONOXIDE | | | | | | | |
| CARBON- | 1.18E−03 | 0.023047 | 0.016764 | 0.016825 | 1.59E−03 | 0.013889 | 0.016736 |
| DIOXIDE | | | | | | | |
| METHANE | 1.74E−03 | 0.162218 | 0.145325 | 0.145904 | 9.72E−04 | 0.18644 | 0.147132 |
| ACETYLENE | 7.11E−04 | 2.28E−03 | 1.57E−03 | 1.57E−03 | 1.54E−03 | 8.87E−04 | 1.55E−03 |
| ETHYLENE | 0.010213 | 0.255987 | 0.190436 | 0.191166 | 8.56E−03 | 0.234707 | 0.192484 |
| ETHANE | 1.36E−04 | 4.62E−03 | 3.53E−03 | 3.54E−03 | 1.08E−04 | 4.41E−03 | 3.57E−03 |
| PROPADIENE | 2.16E−05 | 9.53E−05 | 6.65E−05 | 6.67E−05 | 3.77E−05 | 4.37E−05 | 6.60E−05 |
| METHYL- | 1.54E−07 | 1.07E−06 | 7.55E−07 | 7.57E−07 | 3.66E−07 | 3.84E−07 | 7.46E−07 |
| ACETYLENE | | | | | | | |
| PROPYLENE | 1.51E−04 | 2.45E−03 | 1.78E−03 | 1.79E−03 | 2.30E−04 | 1.33E−03 | 1.78E−03 |
| PROPANE | 2.50E−06 | 6.55E−05 | 4.91E−05 | 4.93E−05 | 3.80E−06 | 3.66E−05 | 4.89E−05 |
| 1,3-BUTADIENE | 3.20E−04 | 9.51E−04 | 6.65E−04 | 6.65E−04 | 7.81E−04 | 3.23E−04 | 6.54E−04 |
| ISOBUTYLENE | 8.70E−06 | 6.84E−05 | 4.84E−05 | 4.86E−05 | 1.61E−05 | 3.03E−05 | 4.80E−05 |
| CIS-2-BUTENE | 6.69E−06 | 5.31E−05 | 3.69E−05 | 3.70E−05 | 1.11E−05 | 2.59E−05 | 3.67E−05 |
| TRANS-2-BUTENE | 5.13E−06 | 1.14E−05 | 7.94E−06 | 7.96E−06 | 2.95E−06 | 1.43E−05 | 8.15E−06 |
| 1-HEXENE | 1.27E−04 | 1.86E−04 | 1.30E−04 | 1.28E−04 | 4.32E−04 | 4.69E−05 | 1.26E−04 |
| N-HEXANE | 2.47E−04 | 8.60E−04 | 5.93E−04 | 5.92E−04 | 7.35E−04 | 2.47E−04 | 5.82E−04 |
| 1-HEXADECENE | 2.74E−04 | 9.53E−07 | 6.24E−07 | 4.16E−09 | 1.55E−04 | 1.36E−08 | 4.45E−09 |
| N-METHYL-2- | 0.982099 | 5.73E−03 | 4.04E−03 | 1.13E−04 | 0.983257 | 1.76E−04 | 1.15E−04 |
| PYRROLIDONE | | | | | | | |
| Stream (Example 1) | 123 | 126 | 124 | 127 | 128 | 129 | 118 |
| Mole Flow lbmol/hr | | | | | | | |
| HYDROGEN | 2.34E−03 | 5.85E−05 | 2.28E−03 | 5.85E−05 | 5.01E−08 | 3.99E−28 | 0 |
| NITROGEN | 1.18E−04 | 2.96E−06 | 1.15E−04 | 2.96E−06 | 2.82E−09 | 2.84E−30 | 0 |
| OXYGEN | 4.75E−04 | 1.19E−05 | 4.63E−04 | 1.18E−05 | 1.09E−07 | 8.04E−31 | 0 |
| CARBON- | 0.015158 | 3.79E−04 | 0.014779 | 3.78E−04 | 7.00E−07 | 1.66E−25 | 0 |
| MONOXIDE | | | | | | | |
| CARBON- | 0.017533 | 4.38E−04 | 0.017095 | 4.17E−04 | 2.16E−05 | 6.58E−21 | 0 |
| DIOXIDE | | | | | | | |
| METHANE | 0.018554 | 4.64E−04 | 0.01809 | 4.62E−04 | 2.17E−06 | 3.10E−24 | 0 |
| ACETYLENE | 0.010867 | 2.72E−04 | 0.010595 | 1.86E−04 | 8.53E−05 | 3.70E−16 | 0 |
| ETHYLENE | 0.146361 | 3.66E−03 | 0.142702 | 3.56E−03 | 9.90E−05 | 3.99E−20 | 0 |
| ETHANE | 1.89E−03 | 4.71E−05 | 1.84E−03 | 4.63E−05 | 8.91E−07 | 8.76E−23 | 0 |
| PROPADIENE | 3.29E−04 | 8.22E−06 | 3.20E−04 | 6.26E−06 | 1.96E−06 | 9.56E−20 | 0 |
| METHYL- | 2.34E−06 | 5.85E−08 | 2.28E−06 | 4.72E−08 | 1.13E−08 | 9.67E−23 | 0 |
| ACETYLENE | | | | | | | |
| PROPYLENE | 2.26E−03 | 5.64E−05 | 2.20E−03 | 5.27E−05 | 3.72E−06 | 2.08E−21 | 0 |
| PROPANE | 3.67E−05 | 9.17E−07 | 3.58E−05 | 8.80E−07 | 3.70E−08 | 1.90E−24 | 0 |
| 1,3-BUTADIENE | 4.90E−03 | 1.23E−04 | 4.78E−03 | 7.66E−05 | 4.60E−05 | 2.56E−17 | 0 |
| ISOBUTYLENE | 1.32E−04 | 3.30E−06 | 1.29E−04 | 2.80E−06 | 5.00E−07 | 5.59E−21 | 0 |
| CIS-2-BUTENE | 1.01E−04 | 2.54E−06 | 9.89E−05 | 2.19E−06 | 3.48E−07 | 6.08E−21 | 0 |
| TRANS-2-BUTENE | 7.80E−05 | 1.95E−06 | 7.60E−05 | 1.55E−06 | 4.01E−07 | 2.88E−16 | 0 |
| 1-HEXENE | 1.95E−03 | 4.88E−05 | 1.90E−03 | 1.87E−05 | 3.00E−05 | 3.43E−16 | 0 |
| N-HEXANE | 3.78E−03 | 9.44E−05 | 3.68E−03 | 5.93E−05 | 3.51E−05 | 5.55E−18 | 0 |
| 1-HEXADECENE | 4.31E−03 | 1.08E−04 | 4.20E−03 | 6.19E−10 | 5.07E−06 | 1.03E−04 | 0 |
| N-METHYL-2- | 15.40127 | 0.385032 | 15.05436 | 1.46E−05 | 0.036948 | 0.348069 | 0.038122 |
| PYRROLIDONE | | | | | | | |
| Mole Frac | | | | | | | |
| HYDROGEN | 1.50E−04 | 1.50E−04 | 1.49E−04 | 0.010916 | 1.34E−06 | 1.15E−27 | 0 |
| NITROGEN | 7.57E−06 | 7.57E−06 | 7.55E−06 | 5.52E−04 | 7.55E−08 | 8.16E−30 | 0 |
| OXYGEN | 3.04E−05 | 3.04E−05 | 3.03E−05 | 2.20E−03 | 2.94E−06 | 2.31E−30 | 0 |
| CARBON- | 9.70E−04 | 9.70E−04 | 9.67E−04 | 0.070594 | 1.88E−05 | 4.77E−25 | 0 |
| MONOXIDE | | | | | | | |
| CARBON- | 1.12E−03 | 1.12E−03 | 1.12E−03 | 0.077768 | 5.81E−04 | 1.89E−20 | 0 |
| DIOXIDE | | | | | | | |
| METHANE | 1.19E−03 | 1.19E−03 | 1.18E−03 | 0.086163 | 5.83E−05 | 8.89E−24 | 0 |
| ACETYLENE | 6.95E−04 | 6.95E−04 | 6.93E−04 | 0.034786 | 2.29E−03 | 1.06E−15 | 0 |
| ETHYLENE | 9.36E−03 | 9.36E−03 | 9.34E−03 | 0.664415 | 2.66E−03 | 1.14E−19 | 0 |
| ETHANE | 1.21E−04 | 1.21E−04 | 1.20E−04 | 8.63E−03 | 2.39E−05 | 2.52E−22 | 0 |
| PROPADIENE | 2.10E−05 | 2.10E−05 | 2.10E−05 | 1.17E−03 | 5.25E−05 | 2.75E−19 | 0 |
| METHYL- | 1.50E−07 | 1.50E−07 | 1.49E−07 | 8.81E−06 | 3.03E−07 | 2.78E−22 | 0 |
| ACETYLENE | | | | | | | |
| PROPYLENE | 1.44E−04 | 1.44E−04 | 1.44E−04 | 9.83E−03 | 9.97E−05 | 5.97E−21 | 0 |
| PROPANE | 2.35E−06 | 2.35E−06 | 2.34E−06 | 1.64E−04 | 9.92E−07 | 5.46E−24 | 0 |
| 1,3-BUTADIENE | 3.13E−04 | 3.13E−04 | 3.13E−04 | 0.014288 | 1.23E−03 | 7.36E−17 | 0 |
| ISOBUTYLENE | 8.45E−06 | 8.45E−06 | 8.43E−06 | 5.23E−04 | 1.34E−05 | 1.61E−20 | 0 |
| CIS-2-BUTENE | 6.49E−06 | 6.49E−06 | 6.47E−06 | 4.08E−04 | 9.34E−06 | 1.75E−20 | 0 |
| TRANS-2-BUTENE | 4.99E−06 | 4.99E−06 | 4.97E−06 | 2.89E−04 | 1.07E−05 | 8.28E−16 | 0 |
| 1-HEXENE | 1.25E−04 | 1.25E−04 | 1.24E−04 | 3.50E−03 | 8.06E−04 | 9.85E−16 | 0 |
| N-HEXANE | 2.42E−04 | 2.42E−04 | 2.41E−04 | 0.011073 | 9.40E−04 | 1.59E−17 | 0 |
| 1-HEXADECENE | 2.76E−04 | 2.76E−04 | 2.75E−04 | 1.16E−07 | 1.36E−04 | 2.95E−04 | 0 |
| N-METHYL-2- | 0.985213 | 0.985213 | 0.98525 | 2.73E−03 | 0.991068 | 0.999705 | 1 |
| PYRROLIDONE | | | | | | | |

| Stream (Example 1) | 100 | 101 | 102 | 103 | 104 | 107 | 109 | 108 |
|---|---|---|---|---|---|---|---|---|
| Mass Flow lb/hr | | | | | | | | |
| HYDROGEN | 7.721192 | 6.176954 | 1.544238 | 6.154866 | 0.02669 | 1.570928 | 0.657313 | 0.036216 |
| NITROGEN | 2.207047 | 1.765637 | 0.441409 | 1.760533 | 8.34E−03 | 0.449747 | 0.426644 | 0.023104 |
| OXYGEN | 0.517188 | 0.41375 | 0.103438 | 0.339725 | 0.08885 | 0.192288 | 0.168163 | 0.024126 |
| CARBON- | 75.73806 | 60.59045 | 15.14761 | 60.45158 | 0.552828 | 15.70044 | 14.22541 | 1.475029 |
| MONOXIDE | | | | | | | | |
| CARBON- | 4.428842 | 3.543074 | 0.885768 | 3.355857 | 0.939556 | 1.825324 | 0.971454 | 0.85387 |
| DIOXIDE | | | | | | | | |
| METHANE | 16.74716 | 13.39772 | 3.349431 | 13.36293 | 0.325001 | 3.674432 | 3.103213 | 0.571219 |
| ACETYLENE | 11.21 | 8.967999 | 2.242 | 0.111987 | 9.131884 | 11.37388 | 0.053752 | 0.287009 |
| ETHYLENE | 1.00345 | 0.80276 | 0.20069 | 4.694904 | 0.111193 | 0.311883 | 7.043609 | 4.787468 |
| ETHANE | 0 | 0 | 0 | 0.055284 | 3.12E−16 | 3.12E−16 | 0.140046 | 0.070114 |
| PROPADIENE | 0.207169 | 0.165735 | 0.041434 | 8.14E−03 | 0.170431 | 0.211865 | 3.50E−03 | 0.013447 |
| METHYL- | 1.70E−03 | 1.36E−03 | 3.40E−04 | 8.66E−05 | 1.37E−03 | 1.71E−03 | 3.98E−05 | 9.67E−05 |
| ACETYLENE | | | | | | | | |
| PROPYLENE | 0 | 0 | 0 | 0.092523 | 3.59E−08 | 3.59E−08 | 0.0989 | 0.102986 |
| PROPANE | 0 | 0 | 0 | 1.58E−03 | 5.04E−13 | 5.04E−13 | 2.85E−03 | 1.85E−03 |
| 1,3-BUTADIENE | 0 | 0 | 0 | 0.084502 | 0.173944 | 0.173944 | 0.047272 | 0.268442 |
| ISOBUTYLENE | 0 | 0 | 0 | 6.99E−03 | 2.34E−04 | 2.34E−04 | 3.57E−03 | 7.69E−03 |
| CIS-2-BUTENE | 0 | 0 | 0 | 5.49E−03 | 6.07E−05 | 6.07E−05 | 2.72E−03 | 5.92E−03 |
| TRANS-2-BUTENE | 0 | 0 | 0 | 3.40E−03 | 8.61E−04 | 8.61E−04 | 5.85E−04 | 4.44E−03 |
| 1-HEXENE | 0.218195 | 0.174556 | 0.043639 | 0.019571 | 0.31506 | 0.358699 | 0.014333 | 0.165017 |
| N-HEXANE | 0 | 0 | 0 | 0.103786 | 0.213437 | 0.213437 | 0.067054 | 0.330028 |
| 1-HEXADECENE | 0 | 0 | 0 | 1.71E−05 | 0.942723 | 0.942723 | 1.83E−04 | 0.943765 |
| N-METHYL-2- | 0 | 0 | 0 | 0.1051 | 1492.273 | 1492.273 | 0.526909 | 1491.746 |
| PYRROLIDONE | | | | | | | | |
| Mass Frac | | | | | | | | |
| HYDROGEN | 0.064343 | 0.064343 | 0.064343 | 0.067846 | 1.77E−05 | 1.03E−03 | 0.023852 | 2.41E−05 |
| NITROGEN | 0.018392 | 0.018392 | 0.018392 | 0.019406 | 5.54E−06 | 2.94E−04 | 0.015482 | 1.54E−05 |
| OXYGEN | 4.31E−03 | 4.31E−03 | 4.31E−03 | 3.74E−03 | 5.90E−05 | 1.26E−04 | 6.10E−03 | 1.61E−05 |
| CARBON- | 0.631151 | 0.631151 | 0.631151 | 0.666362 | 3.67E−04 | 0.010267 | 0.516208 | 9.82E−04 |
| MONOXIDE | | | | | | | | |
| CARBON- | 0.036907 | 0.036907 | 0.036907 | 0.036992 | 6.24E−04 | 1.19E−03 | 0.035252 | 5.69E−04 |
| DIOXIDE | | | | | | | | |
| METHANE | 0.13956 | 0.13956 | 0.13956 | 0.147301 | 2.16E−04 | 2.40E−03 | 0.112609 | 3.80E−04 |
| ACETYLENE | 0.093417 | 0.093417 | 0.093417 | 1.23E−03 | 6.07E−03 | 7.44E−03 | 1.95E−03 | 1.91E−04 |
| ETHYLENE | 8.36E−03 | 8.36E−03 | 8.36E−03 | 0.051752 | 7.39E−05 | 2.04E−04 | 0.255597 | 3.19E−03 |
| ETHANE | 0 | 0 | 0 | 6.09E−04 | 2.07E−19 | 2.04E−19 | 5.08E−03 | 4.67E−05 |
| PROPADIENE | 1.73E−03 | 1.73E−03 | 1.73E−03 | 8.98E−05 | 1.13E−04 | 1.39E−04 | 1.27E−04 | 8.95E−06 |
| METHYL- | 1.42E−05 | 1.42E−05 | 1.42E−05 | 9.54E−07 | 9.08E−07 | 1.12E−06 | 1.44E−06 | 6.44E−08 |
| ACETYLENE | | | | | | | | |
| PROPYLENE | 0 | 0 | 0 | 1.02E−03 | 2.38E−11 | 2.35E−11 | 3.59E−03 | 6.86E−05 |
| PROPANE | 0 | 0 | 0 | 1.74E−05 | 3.35E−16 | 3.29E−16 | 1.04E−04 | 1.23E−06 |
| 1,3-BUTADIENE | 0 | 0 | 0 | 9.31E−04 | 1.16E−04 | 1.14E−04 | 1.72E−03 | 1.79E−04 |
| ISOBUTYLENE | 0 | 0 | 0 | 7.71E−05 | 1.56E−07 | 1.53E−07 | 1.30E−04 | 5.12E−06 |
| CIS-2-BUTENE | 0 | 0 | 0 | 6.05E−05 | 4.04E−08 | 3.97E−08 | 9.88E−05 | 3.94E−06 |
| TRANS-2-BUTENE | 0 | 0 | 0 | 3.75E−05 | 5.72E−07 | 5.63E−07 | 2.12E−05 | 2.96E−06 |
| 1-HEXENE | 1.82E−03 | 1.82E−03 | 1.82E−03 | 2.16E−04 | 2.09E−04 | 2.35E−04 | 5.20E−04 | 1.10E−04 |
| N-HEXANE | 0 | 0 | 0 | 1.14E−03 | 1.42E−04 | 1.40E−04 | 2.43E−03 | 2.20E−04 |
| 1-HEXADECENE | 0 | 0 | 0 | 1.89E−07 | 6.26E−04 | 6.16E−04 | 6.65E−06 | 6.28E−04 |
| N-METHYL-2- | 0 | 0 | 0 | 1.16E−03 | 0.991362 | 0.975804 | 0.01912 | 0.99336 |
| PYRROLIDONE | | | | | | | | |
| Total Flow lbmol/hr | 8.247701 | 6.59816 | 1.64954 | 6.374322 | 15.50358 | 17.15312 | 1.337484 | 15.37731 |
| Total Flow lb/hr | 120 | 96 | 24 | 90.71886 | 1505.275 | 1529.275 | 27.55753 | 1501.718 |
| Total Flow cuft/hr | 164.2334 | 131.3867 | 32.84669 | 276.4045 | 31.31621 | 115.6808 | 40.03523 | 32.69165 |
| Temperature F. | 85 | 85 | 85 | 102.9394 | 106.4619 | 187.6732 | 229.9345 | 229.9345 |
| Pressure psia | 294.6959 | 294.6959 | 294.6959 | 139.6959 | 141.6959 | 140.6959 | 247.6959 | 247.6959 |
| Density lb/cuft | 0.730667 | 0.730667 | 0.730667 | 0.328211 | 48.06697 | 13.21978 | 0.688332 | 45.93582 |
| Average MW | 14.54951 | 14.54951 | 14.54951 | 14.23192 | 97.09209 | 89.15434 | 20.60401 | 97.65801 |

| Stream (Example 1) | 114 | 112 | 113 | 117 | 116 | 121 | 122 |
|---|---|---|---|---|---|---|---|
| Mass Flow lb/hr | | | | | | | |
| HYDROGEN | 0.017003 | 0.019213 | 0.676526 | 0.676523 | 2.88E−06 | 0.012286 | 0.688809 |
| NITROGEN | 0.010733 | 0.012371 | 0.439014 | 0.439012 | 2.20E−06 | 7.42E−03 | 0.446431 |
| OXYGEN | 0.015865 | 8.26E−03 | 0.176423 | 0.176355 | 6.87E−05 | 7.28E−04 | 0.177083 |
| CARBON- | 0.922081 | 0.552947 | 14.77836 | 14.77822 | 1.42E−04 | 0.497646 | 15.27586 |
| MONOXIDE | | | | | | | |
| CARBON- | 0.797719 | 0.056151 | 1.027605 | 1.027216 | 3.89E−04 | 0.026483 | 1.053698 |
| DIOXIDE | | | | | | | |
| METHANE | 0.427147 | 0.144072 | 3.247286 | 3.247199 | 8.68E−05 | 0.129584 | 3.376783 |
| ACETYLENE | 0.283723 | 3.29E−03 | 0.057039 | 0.056815 | 2.24E−04 | 1.00E−03 | 0.057816 |
| ETHYLENE | 4.389901 | 0.397567 | 7.441175 | 7.439839 | 1.34E−03 | 0.285267 | 7.725105 |
| ETHANE | 0.062431 | 7.68E−03 | 0.14773 | 0.147712 | 1.81E−05 | 5.75E−03 | 0.15346 |
| PROPADIENE | 0.013236 | 2.11E−04 | 3.71E−03 | 3.71E−03 | 8.41E−06 | 7.59E−05 | 3.78E−03 |
| METHYL- | 9.44E−05 | 2.37E−06 | 4.22E−05 | 4.21E−05 | 8.16E−08 | 6.66E−07 | 4.27E−05 |
| ACETYLENE | | | | | | | |
| PROPYLENE | 0.097268 | 5.72E−03 | 0.104618 | 0.104564 | 5.40E−05 | 2.43E−03 | 0.106991 |
| PROPANE | 1.69E−03 | 1.60E−04 | 3.01E−03 | 3.01E−03 | 9.34E−07 | 6.99E−05 | 3.08E−03 |
| 1,3-BUTADIENE | 0.265595 | 2.85E−03 | 0.050119 | 0.049884 | 2.35E−04 | 7.58E−04 | 0.050642 |
| ISOBUTYLENE | 7.48E−03 | 2.12E−04 | 3.78E−03 | 3.78E−03 | 5.03E−06 | 7.36E−05 | 3.85E−03 |
| CIS-2-BUTENE | 5.75E−03 | 1.65E−04 | 2.89E−03 | 2.88E−03 | 3.46E−06 | 6.30E−05 | 2.95E−03 |
| TRANS-2-BUTENE | 4.41E−03 | 3.55E−05 | 6.20E−04 | 6.19E−04 | 9.23E−07 | 3.49E−05 | 6.54E−04 |
| 1-HEXENE | 0.164148 | 8.69E−04 | 0.015201 | 0.014999 | 2.02E−04 | 1.71E−04 | 0.01517 |
| N-HEXANE | 0.325924 | 4.10E−03 | 0.071158 | 0.070805 | 3.53E−04 | 9.23E−04 | 0.071728 |
| 1-HEXADECENE | 0.943753 | 1.18E−05 | 1.95E−04 | 1.29E−06 | 1.94E−04 | 1.33E−07 | 1.43E−06 |
| N-METHYL-2- | 1491.714 | 0.03142 | 0.558329 | 0.015517 | 0.542811 | 7.57E−04 | 0.016274 |
| PYRROLIDONE | | | | | | | |
| Mass Frac | | | | | | | |
| HYDROGEN | 1.13E−05 | 0.015403 | 0.023487 | 0.02394 | 5.27E−06 | 0.012646 | 0.023565 |
| NITROGEN | 7.15E−06 | 9.92E−03 | 0.015241 | 0.015535 | 4.02E−06 | 7.64E−03 | 0.015273 |
| OXYGEN | 1.06E−05 | 6.62E−03 | 6.12E−03 | 6.24E−03 | 1.26E−04 | 7.49E−04 | 6.06E−03 |
| CARBON- | 6.15E−04 | 0.443313 | 0.513051 | 0.522962 | 2.61E−04 | 0.512236 | 0.522605 |
| MONOXIDE | | | | | | | |
| CARBON- | 5.32E−04 | 0.045018 | 0.035675 | 0.03635 | 7.13E−04 | 0.027259 | 0.036048 |
| DIOXIDE | | | | | | | |
| METHANE | 2.85E−04 | 0.115507 | 0.112734 | 0.11491 | 1.59E−04 | 0.133384 | 0.115524 |
| ACETYLENE | 1.89E−04 | 2.64E−03 | 1.98E−03 | 2.01E−03 | 4.10E−04 | 1.03E−03 | 1.98E−03 |
| ETHYLENE | 2.93E−03 | 0.31874 | 0.258331 | 0.263276 | 2.45E−03 | 0.29363 | 0.264285 |
| ETHANE | 4.16E−05 | 6.16E−03 | 5.13E−03 | 5.23E−03 | 3.32E−05 | 5.92E−03 | 5.25E−03 |
| PROPADIENE | 8.82E−06 | 1.69E−04 | 1.29E−04 | 1.31E−04 | 1.54E−05 | 7.81E−05 | 1.29E−04 |
| METHYL- | 6.29E−08 | 1.90E−06 | 1.46E−06 | 1.49E−06 | 1.49E−07 | 6.86E−07 | 1.46E−06 |
| ACETYLENE | | | | | | | |
| PROPYLENE | 6.48E−05 | 4.58E−03 | 3.63E−03 | 3.70E−03 | 9.89E−05 | 2.50E−03 | 3.66E−03 |
| PROPANE | 1.12E−06 | 1.28E−04 | 1.05E−04 | 1.07E−04 | 1.71E−06 | 7.19E−05 | 1.05E−04 |
| 1,3-BUTADIENE | 1.77E−04 | 2.28E−03 | 1.74E−03 | 1.77E−03 | 4.31E−04 | 7.80E−04 | 1.73E−03 |
| ISOBUTYLENE | 4.98E−06 | 1.70E−04 | 1.31E−04 | 1.34E−04 | 9.21E−06 | 7.58E−05 | 1.32E−04 |
| CIS-2-BUTENE | 3.83E−06 | 1.32E−04 | 1.00E−04 | 1.02E−04 | 6.33E−06 | 6.48E−05 | 1.01E−04 |
| TRANS-2-BUTENE | 2.94E−06 | 2.85E−05 | 2.15E−05 | 2.19E−05 | 1.69E−06 | 3.59E−05 | 2.24E−05 |
| 1-HEXENE | 1.09E−04 | 6.96E−04 | 5.28E−04 | 5.31E−04 | 3.70E−04 | 1.76E−04 | 5.19E−04 |
| N-HEXANE | 2.17E−04 | 3.29E−03 | 2.47E−03 | 2.51E−03 | 6.46E−04 | 9.51E−04 | 2.45E−03 |
| 1-HEXADECENE | 6.29E−04 | 9.49E−06 | 6.77E−06 | 4.58E−08 | 3.55E−04 | 1.37E−07 | 4.88E−08 |
| N-METHYL-2- | 0.994165 | 0.02519 | 0.019383 | 5.49E−04 | 0.993906 | 7.79E−04 | 5.57E−04 |
| PYRROLIDONE | | | | | | | |
| Total Flow lbmol/hr | 15.32195 | 0.055361 | 1.392844 | 1.387276 | 5.57E−03 | 0.043325 | 1.4306 |
| Total Flow lb/hr | 1500.47 | 1.247307 | 28.80484 | 28.2587 | 0.54614 | 0.971517 | 29.23022 |
| Total Flow cuft/hr | 32.66701 | 2.486317 | 62.48839 | 89.4793 | 0.011257 | 2.84856 | 92.32953 |
| Temperature F. | 230.4519 | 230.4519 | 228.1737 | 81.6632 | 81.6632 | 92.72012 | 82.00916 |
| Pressure psia | 164.6959 | 164.6959 | 164.6959 | 89.69595 | 89.69595 | 89.69595 | 89.69595 |
| Density lb/cuft | 45.93228 | 0.501669 | 0.460963 | 0.315813 | 48.51506 | 0.341055 | 0.316586 |
| Average MW | 97.92946 | 22.53053 | 20.68059 | 20.36992 | 98.07046 | 22.42415 | 20.43214 |

| Stream (Example 1) | 123 | 126 | 124 | 127 | 128 | 129 | 118 |
|---|---|---|---|---|---|---|---|
| Mass Flow lb/hr | | | | | | | |
| HYDROGEN | 4.72E−03 | 1.18E−04 | 4.60E−03 | 1.18E−04 | 1.01E−07 | 8.05E−28 | 0 |
| NITROGEN | 3.32E−03 | 8.29E−05 | 3.23E−03 | 8.28E−05 | 7.89E−08 | 7.95E−29 | 0 |
| OXYGEN | 0.015205 | 3.80E−04 | 0.014825 | 3.77E−04 | 3.50E−06 | 2.57E−29 | 0 |
| CARBON- | 0.424577 | 0.010614 | 0.413963 | 0.010595 | 1.96E−05 | 4.65E−24 | 0 |
| MONOXIDE | | | | | | | |
| CARBON- | 0.77163 | 0.019291 | 0.75234 | 0.018338 | 9.53E−04 | 2.89E−19 | 0 |
| DIOXIDE | | | | | | | |
| METHANE | 0.297651 | 7.44E−03 | 0.29021 | 7.41E−03 | 3.49E−05 | 4.97E−23 | 0 |
| ACETYLENE | 0.282946 | 7.07E−03 | 0.275872 | 4.85E−03 | 2.22E−03 | 9.64E−15 | 0 |
| ETHYLENE | 4.105986 | 0.10265 | 4.003336 | 0.099871 | 2.78E−03 | 1.12E−18 | 0 |
| ETHANE | 0.056701 | 1.42E−03 | 0.055284 | 1.39E−03 | 2.68E−05 | 2.64E−21 | 0 |
| PROPADIENE | 0.013168 | 3.29E−04 | 0.012839 | 2.51E−04 | 7.84E−05 | 3.83E−18 | 0 |
| METHYL- | 9.38E−05 | 2.34E−06 | 9.14E−05 | 1.89E−06 | 4.53E−07 | 3.87E−21 | 0 |
| ACETYLENE | | | | | | | |
| PROPYLENE | 0.094895 | 2.37E−03 | 0.092523 | 2.22E−03 | 1.56E−04 | 8.75E−20 | 0 |
| PROPANE | 1.62E−03 | 4.04E−05 | 1.58E−03 | 3.88E−05 | 1.63E−06 | 8.39E−23 | 0 |
| 1,3-BUTADIENE | 0.265073 | 6.63E−03 | 0.258446 | 4.14E−03 | 2.49E−03 | 1.39E−15 | 0 |
| ISOBUTYLENE | 7.41E−03 | 1.85E−04 | 7.23E−03 | 1.57E−04 | 2.81E−05 | 3.14E−19 | 0 |
| CIS-2-BUTENE | 5.69E−03 | 1.42E−04 | 5.55E−03 | 1.23E−04 | 1.95E−05 | 3.41E−19 | 0 |
| TRANS-2-BUTENE | 4.37E−03 | 1.09E−04 | 4.26E−03 | 8.69E−05 | 2.25E−05 | 1.62E−14 | 0 |
| 1-HEXENE | 0.16418 | 4.10E−03 | 0.160075 | 1.58E−03 | 2.53E−03 | 2.89E−14 | 0 |
| N-HEXANE | 0.325356 | 8.13E−03 | 0.317222 | 5.11E−03 | 3.02E−03 | 4.78E−16 | 0 |
| 1-HEXADECENE | 0.966913 | 2.42E−02 | 0.94274 | 1.39E−07 | 1.14E−03 | 0.023036 | 0 |
| N-METHYL-2- | 1526.768 | 38.1692 | 1492.378 | 1.45E−03 | 3.662746 | 34.505 | 3.779109 |
| PYRROLIDONE | | | | | | | |
| Mass Frac | | | | | | | |
| HYDROGEN | 3.08E−06 | 3.08E−06 | 3.07E−06 | 7.45E−04 | 2.74E−08 | 2.33E−29 | 0 |
| NITROGEN | 2.16E−06 | 2.16E−06 | 2.16E−06 | 5.24E−04 | 2.14E−08 | 2.30E−30 | 0 |
| OXYGEN | 9.91E−06 | 9.91E−06 | 9.88E−06 | 2.38E−03 | 9.52E−07 | 7.45E−31 | 0 |
| CARBON- | 2.77E−04 | 2.77E−04 | 2.76E−04 | 0.066977 | 5.33E−06 | 1.35E−25 | 0 |
| MONOXIDE | | | | | | | |
| CARBON- | 5.03E−04 | 5.03E−04 | 5.02E−04 | 0.115927 | 2.59E−04 | 8.38E−21 | 0 |
| DIOXIDE | | | | | | | |
| METHANE | 1.94E−04 | 1.94E−04 | 1.93E−04 | 0.046821 | 9.48E−06 | 1.44E−24 | 0 |
| ACETYLENE | 1.84E−04 | 1.84E−04 | 1.84E−04 | 0.030679 | 6.04E−04 | 2.79E−16 | 0 |
| ETHYLENE | 2.68E−03 | 2.68E−03 | 2.67E−03 | 0.631347 | 7.55E−04 | 3.24E−20 | 0 |
| ETHANE | 3.69E−05 | 3.69E−05 | 3.69E−05 | 8.79E−03 | 7.28E−06 | 7.63E−23 | 0 |
| PROPADIENE | 8.58E−06 | 8.58E−06 | 8.56E−06 | 1.59E−03 | 2.13E−05 | 1.11E−19 | 0 |
| METHYL- | 6.11E−08 | 6.11E−08 | 6.10E−08 | 1.20E−05 | 1.23E−07 | 1.12E−22 | 0 |
| ACETYLENE | | | | | | | |
| PROPYLENE | 6.18E−05 | 6.18E−05 | 6.17E−05 | 0.014008 | 4.25E−05 | 2.53E−21 | 0 |
| PROPANE | 1.05E−06 | 1.05E−06 | 1.05E−06 | 2.45E−04 | 4.43E−07 | 2.43E−24 | 0 |
| 1,3-BUTADIENE | 1.73E−04 | 1.73E−04 | 1.72E−04 | 0.026178 | 6.76E−04 | 4.02E−17 | 0 |
| ISOBUTYLENE | 4.83E−06 | 4.83E−06 | 4.82E−06 | 9.94E−04 | 7.63E−06 | 9.09E−21 | 0 |
| CIS-2-BUTENE | 3.71E−06 | 3.71E−06 | 3.70E−06 | 7.76E−04 | 5.31E−06 | 9.89E−21 | 0 |
| TRANS-2-BUTENE | 2.85E−06 | 2.85E−06 | 2.84E−06 | 5.49E−04 | 6.11E−06 | 4.69E−16 | 0 |
| 1-HEXENE | 1.07E−04 | 1.07E−04 | 1.07E−04 | 9.97E−03 | 6.87E−04 | 8.36E−16 | 0 |
| N-HEXANE | 2.12E−04 | 2.12E−04 | 2.11E−04 | 0.032321 | 8.21E−04 | 1.39E−17 | 0 |
| 1-HEXADECENE | 6.30E−04 | 6.30E−04 | 6.28E−04 | 8.78E−07 | 3.09E−04 | 6.67E−04 | 0 |
| N-METHYL-2- | 0.99491 | 0.99491 | 0.994923 | 9.17E−03 | 0.995782 | 0.999333 | 1 |
| PYRROLIDONE | | | | | | | |
| Total Flow lbmol/hr | 15.63243 | 0.390811 | 15.27974 | 5.36E−03 | 0.037281 | 0.348172 | 0.038122 |
| Total Flow lb/hr | 1534.58 | 38.36449 | 1499.994 | 0.158187 | 3.678262 | 34.52804 | 3.779109 |
| Total Flow cuft/hr | 31.74158 | 0.793539 | 31.09663 | 3.378949 | 0.076471 | 0.803035 | 0.077813 |
| Temperature F. | 92.72012 | 92.71976 | 102.4521 | 110 | 110 | 371.0185 | 85 |
| Pressure psia | 89.69595 | 89.69595 | 304.6959 | 9.668387 | 9.668387 | 10.05512 | 29.69595 |
| Density lb/cuft | 48.34604 | 48.34604 | 48.23655 | 0.046815 | 48.10003 | 42.99691 | 48.56632 |
| Average MW | 98.16639 | 98.16639 | 98.1688 | 29.52315 | 98.66333 | 99.16954 | 99.1326 |

TABLE 2

| Stream (Example 2) | 100 | 101 | 102 | 103 | 104 | 107 | 109 | 108 |
|---|---|---|---|---|---|---|---|---|
| Mole Flow lbmol/hr | | | | | | | | |
| HYDROGEN | 3.830185 | 3.064148 | 0.766037 | 3.051202 | 0.013131 | 0.779168 | 0.32602 | 0.017981 |
| NITROGEN | 0.078785 | 0.063028 | 0.015757 | 0.062741 | 2.97E−04 | 0.016054 | 0.015228 | 8.26E−04 |
| OXYGEN | 0.016163 | 0.01293 | 3.23E−03 | 0.010075 | 3.02E−03 | 6.26E−03 | 5.47E−03 | 7.87E−04 |
| CARBON- | 2.703926 | 2.163141 | 0.540785 | 2.145023 | 0.01969 | 0.560475 | 0.507752 | 0.052723 |
| MONOXIDE | | | | | | | | |
| CARBON- | 0.100633 | 0.080506 | 0.020127 | 0.070028 | 0.022046 | 0.042172 | 0.022419 | 0.019754 |
| DIOXIDE | | | | | | | | |
| METHANE | 1.043907 | 0.835126 | 0.208782 | 0.817926 | 0.020396 | 0.229177 | 0.193492 | 0.035686 |
| ACETYLENE | 0.430527 | 0.344421 | 0.086105 | 3.84E−03 | 0.350646 | 0.436752 | 2.06E−03 | 0.011025 |
| ETHYLENE | 0.035769 | 0.028615 | 7.15E−03 | 0.096617 | 4.04E−03 | 0.011196 | 0.250859 | 0.170884 |
| ETHANE | 0 | 0 | 0 | 7.68E−04 | 5.66E−18 | 5.66E−18 | 4.65E−03 | 2.33E−03 |
| PROPADIENE | 5.17E−03 | 4.14E−03 | 1.03E−03 | 1.79E−04 | 4.25E−03 | 5.29E−03 | 8.72E−05 | 3.36E−04 |
| METHYL- | 4.25E−05 | 3.40E−05 | 8.50E−06 | 1.79E−06 | 3.43E−05 | 4.28E−05 | 9.95E−07 | 2.43E−06 |
| ACETYLENE | | | | | | | | |
| PROPYLENE | 0 | 0 | 0 | 1.61E−03 | 1.02E−09 | 1.02E−09 | 2.35E−03 | 2.45E−03 |
| PROPANE | 0 | 0 | 0 | 2.21E−05 | 1.15E−14 | 1.15E−14 | 6.47E−05 | 4.19E−05 |
| 1,3-BUTADIENE | 0 | 0 | 0 | 1.39E−03 | 3.13E−03 | 3.13E−03 | 8.58E−04 | 4.89E−03 |
| ISOBUTYLENE | 0 | 0 | 0 | 1.08E−04 | 5.24E−06 | 5.24E−06 | 6.39E−05 | 1.38E−04 |
| CIS-2-BUTENE | 0 | 0 | 0 | 8.38E−05 | 1.42E−06 | 1.42E−06 | 4.85E−05 | 1.06E−04 |
| TRANS-2-BUTENE | 0 | 0 | 0 | 5.29E−05 | 1.44E−05 | 1.44E−05 | 1.03E−05 | 7.83E−05 |
| 1-HEXENE | 2.59E−03 | 2.07E−03 | 5.19E−04 | 2.12E−04 | 3.73E−03 | 4.25E−03 | 1.69E−04 | 1.95E−03 |
| N-HEXANE | 0 | 0 | 0 | 1.07E−03 | 2.41E−03 | 2.41E−03 | 7.65E−04 | 3.77E−03 |
| 1-HEXADECENE | 0 | 0 | 0 | 6.29E−08 | 4.10E−03 | 4.10E−03 | 7.94E−07 | 4.11E−03 |
| N-METHYL-2- | 0 | 0 | 0 | 9.27E−04 | 15.08358 | 15.08358 | 5.31E−03 | 15.07828 |
| PYRROLIDONE | | | | | | | | |
| Mole Frac | | | | | | | | |
| HYDROGEN | 0.464394 | 0.464394 | 0.464394 | 0.487112 | 8.45E−04 | 0.045342 | 0.243722 | 1.17E−03 |
| NITROGEN | 9.55E−03 | 9.55E−03 | 9.55E−03 | 1.00E−02 | 1.91E−05 | 9.34E−04 | 0.011384 | 5.36E−05 |
| OXYGEN | 1.96E−03 | 1.96E−03 | 1.96E−03 | 1.61E−03 | 1.95E−04 | 3.64E−04 | 4.09E−03 | 5.11E−05 |
| CARBON- | 0.32784 | 0.32784 | 0.32784 | 0.342444 | 1.27E−03 | 0.032616 | 0.379578 | 3.42E−03 |
| MONOXIDE | | | | | | | | |
| CARBON- | 0.012201 | 0.012201 | 0.012201 | 0.01118 | 1.42E−03 | 2.45E−03 | 0.01676 | 1.28E−03 |
| DIOXIDE | | | | | | | | |
| METHANE | 0.12657 | 0.12657 | 0.12657 | 0.130579 | 1.31E−03 | 0.013337 | 0.144648 | 2.32E−03 |
| ACETYLENE | 0.0522 | 0.0522 | 0.0522 | 6.12E−04 | 0.022572 | 0.025416 | 1.54E−03 | 7.16E−04 |
| ETHYLENE | 4.34E−03 | 4.34E−03 | 4.34E−03 | 0.015425 | 2.60E−04 | 6.52E−04 | 0.187534 | 0.01109 |
| ETHANE | 0 | 0 | 0 | 1.23E−04 | 3.65E−19 | 3.30E−19 | 3.48E−03 | 1.52E−04 |
| PROPADIENE | 6.27E−04 | 6.27E−04 | 6.27E−04 | 2.86E−05 | 2.74E−04 | 3.08E−04 | 6.52E−05 | 2.18E−05 |
| METHYL- | 5.15E−06 | 5.15E−06 | 5.15E−06 | 2.85E−07 | 2.21E−06 | 2.49E−06 | 7.44E−07 | 1.57E−07 |
| ACETYLENE | | | | | | | | |
| PROPYLENE | 0 | 0 | 0 | 2.56E−04 | 6.54E−11 | 5.91E−11 | 1.75E−03 | 1.59E−04 |
| PROPANE | 0 | 0 | 0 | 3.53E−06 | 7.39E−16 | 6.68E−16 | 4.83E−05 | 2.72E−06 |
| 1,3-BUTADIENE | 0 | 0 | 0 | 2.22E−04 | 2.01E−04 | 1.82E−04 | 6.42E−04 | 3.17E−04 |
| ISOBUTYLENE | 0 | 0 | 0 | 1.72E−05 | 3.37E−07 | 3.05E−07 | 4.78E−05 | 8.95E−06 |
| CIS-2-BUTENE | 0 | 0 | 0 | 1.34E−05 | 9.13E−08 | 8.25E−08 | 3.63E−05 | 6.86E−06 |
| TRANS-2-BUTENE | 0 | 0 | 0 | 8.44E−06 | 9.25E−07 | 8.36E−07 | 7.69E−06 | 5.08E−06 |
| 1-HEXENE | 3.14E−04 | 3.14E−04 | 3.14E−04 | 3.38E−05 | 2.40E−04 | 2.47E−04 | 1.27E−04 | 1.27E−04 |
| N-HEXANE | 0 | 0 | 0 | 1.70E−04 | 1.55E−04 | 1.40E−04 | 5.72E−04 | 2.45E−04 |
| 1-HEXADECENE | 0 | 0 | 0 | 1.00E−08 | 2.64E−04 | 2.39E−04 | 5.94E−07 | 2.66E−04 |
| N-METHYL-2- | 0 | 0 | 0 | 1.48E−04 | 0.970971 | 0.877765 | 3.97E−03 | 0.978591 |
| PYRROLIDONE | | | | | | | | |

| Stream (Example 2) | 114 | 112 | 113 | 117 | 116 | 121 | 122 |
|---|---|---|---|---|---|---|---|
| Mole Flow lbmol/hr | | | | | | | |
| HYDROGEN | 8.44E−03 | 9.54E−03 | 0.335562 | 0.335562 | 2.61E−07 | 8.25E−03 | 0.34381 |
| NITROGEN | 3.84E−04 | 4.42E−04 | 0.01567 | 0.01567 | 1.47E−08 | 3.74E−04 | 0.016044 |
| OXYGEN | 5.18E−04 | 2.69E−04 | 5.74E−03 | 5.74E−03 | 9.03E−08 | 3.45E−04 | 6.08E−03 |
| CARBON- | 0.032954 | 0.019769 | 0.527521 | 0.52752 | 9.56E−07 | 0.031343 | 0.558863 |
| MONOXIDE | | | | | | | |
| CARBON- | 0.018455 | 1.30E−03 | 0.023717 | 0.023715 | 1.85E−06 | 6.59E−03 | 0.030309 |
| DIOXIDE | | | | | | | |
| METHANE | 0.026686 | 9.00E−03 | 0.202491 | 0.20249 | 1.07E−06 | 2.34E−02 | 0.225899 |
| ACETYLENE | 0.010899 | 1.26E−04 | 2.19E−03 | 2.18E−03 | 1.88E−06 | 5.83E−04 | 2.77E−03 |
| ETHYLENE | 0.156698 | 0.014185 | 0.265045 | 0.265035 | 9.59E−06 | 0.082816 | 0.347851 |
| ETHANE | 2.08E−03 | 2.56E−04 | 4.91E−03 | 4.91E−03 | 1.20E−07 | 1.29E−03 | 6.20E−03 |
| PROPADIENE | 3.30E−04 | 5.27E−06 | 9.25E−05 | 9.24E−05 | 4.44E−08 | 2.77E−05 | 1.20E−04 |
| METHYL- | 2.37E−06 | 5.93E−08 | 1.05E−06 | 1.05E−06 | 4.44E−10 | 2.38E−07 | 1.29E−06 |
| ACETYLENE | | | | | | | |
| PROPYLENE | 2.31E−03 | 1.36E−04 | 2.48E−03 | 2.48E−03 | 2.70E−07 | 6.68E−04 | 3.15E−03 |
| PROPANE | 3.83E−05 | 3.63E−06 | 6.83E−05 | 6.83E−05 | 4.36E−09 | 1.56E−05 | 8.39E−05 |
| 1,3-BUTADIENE | 4.84E−03 | 5.18E−05 | 9.10E−04 | 9.09E−04 | 9.68E−07 | 2.06E−04 | 1.12E−03 |
| ISOBUTYLENE | 1.34E−04 | 3.80E−06 | 6.77E−05 | 6.77E−05 | 1.99E−08 | 1.80E−05 | 8.57E−05 |
| CIS-2-BUTENE | 1.03E−04 | 2.94E−06 | 5.15E−05 | 5.15E−05 | 1.33E−08 | 1.54E−05 | 6.69E−05 |
| TRANS-2-BUTENE | 7.77E−05 | 6.26E−07 | 1.09E−05 | 1.09E−05 | 3.38E−09 | 8.72E−06 | 1.96E−05 |
| 1-HEXENE | 1.94E−03 | 1.03E−05 | 1.80E−04 | 1.79E−04 | 5.62E−07 | 3.04E−05 | 2.09E−04 |
| N-HEXANE | 3.73E−03 | 4.69E−05 | 8.11E−04 | 8.11E−04 | 8.92E−07 | 1.59E−04 | 9.69E−04 |
| 1-HEXADECENE | 4.11E−03 | 5.14E−08 | 8.46E−07 | 2.10E−08 | 8.25E−07 | 8.55E−09 | 2.96E−08 |
| N-METHYL-2- | 15.07796 | 3.17E−04 | 5.62E−03 | 5.42E−04 | 5.08E−03 | 1.12E−04 | 6.55E−04 |
| PYRROLIDONE | | | | | | | |
| Mole Frac | | | | | | | |
| HYDROGEN | 5.50E−04 | 0.172041 | 0.240868 | 0.241752 | 5.13E−05 | 0.052785 | 0.222631 |
| NITROGEN | 2.50E−05 | 7.97E−03 | 0.011248 | 0.01129 | 2.88E−06 | 2.39E−03 | 0.010389 |
| OXYGEN | 3.37E−05 | 4.86E−03 | 4.12E−03 | 4.13E−03 | 1.77E−05 | 2.20E−03 | 3.94E−03 |
| CARBON- | 2.15E−03 | 0.356416 | 0.378656 | 0.380047 | 1.87E−04 | 0.200577 | 0.361887 |
| MONOXIDE | | | | | | | |
| CARBON- | 1.20E−03 | 0.023405 | 0.017024 | 0.017085 | 3.64E−04 | 0.042196 | 0.019626 |
| DIOXIDE | | | | | | | |
| METHANE | 1.74E−03 | 0.162253 | 0.145349 | 0.145882 | 2.10E−04 | 0.149804 | 0.146279 |
| ACETYLENE | 7.10E−04 | 2.27E−03 | 1.57E−03 | 1.57E−03 | 3.69E−04 | 3.73E−03 | 1.79E−03 |
| ETHYLENE | 0.010207 | 0.255746 | 0.19025 | 0.190942 | 1.88E−03 | 0.52998 | 0.225248 |
| ETHANE | 1.35E−04 | 4.61E−03 | 3.52E−03 | 3.54E−03 | 2.36E−05 | 8.26E−03 | 4.01E−03 |
| PROPADIENE | 2.15E−05 | 9.51E−05 | 6.64E−05 | 6.66E−05 | 8.71E−06 | 1.77E−04 | 7.78E−05 |
| METHYL- | 1.54E−07 | 1.07E−06 | 7.57E−07 | 7.59E−07 | 8.72E−08 | 1.52E−06 | 8.37E−07 |
| ACETYLENE | | | | | | | |
| PROPYLENE | 1.51E−04 | 2.45E−03 | 1.78E−03 | 1.79E−03 | 5.30E−05 | 4.27E−03 | 2.04E−03 |
| PROPANE | 2.49E−06 | 6.54E−05 | 4.90E−05 | 4.92E−05 | 8.56E−07 | 9.98E−05 | 5.43E−05 |
| 1,3-BUTADIENE | 3.15E−04 | 9.34E−04 | 6.53E−04 | 6.55E−04 | 1.90E−04 | 1.32E−03 | 7.22E−04 |
| ISOBUTYLENE | 8.73E−06 | 6.86E−05 | 4.86E−05 | 4.88E−05 | 3.90E−06 | 1.15E−04 | 5.55E−05 |
| CIS-2-BUTENE | 6.70E−06 | 5.31E−05 | 3.70E−05 | 3.71E−05 | 2.60E−06 | 9.85E−05 | 4.33E−05 |
| TRANS-2-BUTENE | 5.06E−06 | 1.13E−05 | 7.83E−06 | 7.86E−06 | 6.62E−07 | 5.58E−05 | 1.27E−05 |
| 1-HEXENE | 1.27E−04 | 1.85E−04 | 1.29E−04 | 1.29E−04 | 1.10E−04 | 1.95E−04 | 1.36E−04 |
| N-HEXANE | 2.43E−04 | 8.45E−04 | 5.82E−04 | 5.84E−04 | 1.75E−04 | 1.01E−03 | 6.28E−04 |
| 1-HEXADECENE | 2.67E−04 | 9.26E−07 | 6.07E−07 | 1.51E−08 | 1.62E−04 | 5.47E−08 | 1.91E−08 |
| N-METHYL-2- | 0.982106 | 5.71E−03 | 4.04E−03 | 3.91E−04 | 0.996187 | 7.19E−04 | 4.24E−04 |
| PYRROLIDONE | | | | | | | |
| Stream (Example 2) | 123 | 126 | 124 | 127 | 128 | 129 | 118 |
| Mole Flow lbmol/hr | | | | | | | |
| HYDROGEN | 1.90E−04 | 4.76E−06 | 1.86E−04 | 4.75E−06 | 9.35E−09 | 1.59E−28 | 0 |
| NITROGEN | 9.97E−06 | 2.49E−07 | 9.72E−06 | 2.49E−07 | 5.40E−10 | 5.56E−30 | 0 |
| OXYGEN | 1.73E−04 | 4.34E−06 | 1.69E−04 | 4.23E−06 | 1.10E−07 | 1.11E−28 | 0 |
| CARBON- | 1.61E−03 | 4.03E−05 | 1.57E−03 | 4.01E−05 | 1.69E−07 | 8.30E−26 | 0 |
| MONOXIDE | | | | | | | |
| CARBON- | 0.011864 | 2.97E−04 | 0.011567 | 2.65E−04 | 3.11E−05 | 7.58E−21 | 0 |
| DIOXIDE | | | | | | | |
| METHANE | 3.28E−03 | 8.20E−05 | 3.20E−03 | 8.11E−05 | 8.63E−07 | 2.21E−24 | 0 |
| ACETYLENE | 0.010318 | 2.58E−04 | 0.01006 | 1.27E−04 | 1.31E−04 | 3.80E−16 | 0 |
| ETHYLENE | 0.073892 | 1.85E−03 | 0.072045 | 1.74E−03 | 1.09E−04 | 4.46E−20 | 0 |
| ETHANE | 7.88E−04 | 1.97E−05 | 7.68E−04 | 1.89E−05 | 8.23E−07 | 9.40E−23 | 0 |
| PROPADIENE | 3.03E−04 | 7.57E−06 | 2.95E−04 | 4.44E−06 | 3.13E−06 | 9.98E−20 | 0 |
| METHYL- | 2.13E−06 | 5.32E−08 | 2.08E−06 | 3.45E−08 | 1.87E−08 | 1.02E−22 | 0 |
| ACETYLENE | | | | | | | |
| PROPYLENE | 1.65E−03 | 4.12E−05 | 1.61E−03 | 3.55E−05 | 5.68E−06 | 2.33E−21 | 0 |
| PROPANE | 2.27E−05 | 5.68E−07 | 2.21E−05 | 5.18E−07 | 4.94E−08 | 2.16E−24 | 0 |
| 1,3-BUTADIENE | 4.63E−03 | 1.16E−04 | 4.51E−03 | 4.91E−05 | 6.67E−05 | 2.58E−17 | 0 |
| ISOBUTYLENE | 1.16E−04 | 2.90E−06 | 1.13E−04 | 2.06E−06 | 8.37E−07 | 6.00E−21 | 0 |
| CIS-2-BUTENE | 8.74E−05 | 2.19E−06 | 8.52E−05 | 1.60E−06 | 5.82E−07 | 6.55E−21 | 0 |
| TRANS-2-BUTENE | 6.90E−05 | 1.72E−06 | 6.73E−05 | 1.09E−06 | 6.39E−07 | 3.01E−16 | 0 |
| 1-HEXENE | 1.91E−03 | 4.78E−05 | 1.87E−03 | 1.03E−05 | 3.75E−05 | 3.44E−16 | 0 |
| N-HEXANE | 3.57E−03 | 8.92E−05 | 3.48E−03 | 3.79E−05 | 5.13E−05 | 5.58E−18 | 0 |
| 1-HEXADECENE | 4.21E−03 | 1.05E−04 | 4.10E−03 | 2.69E−10 | 5.05E−06 | 1.00E−04 | 0 |
| N-METHYL-2- | 15.43098 | 0.385775 | 15.08451 | 6.63E−06 | 0.037719 | 0.348049 | 0.039304 |
| PYRROLIDONE | | | | | | | |
| Mole Frac | | | | | | | |
| HYDROGEN | 1.22E−05 | 1.22E−05 | 1.22E−05 | 1.96E−03 | 2.45E−07 | 4.58E−28 | 0 |
| NITROGEN | 6.41E−07 | 6.41E−07 | 6.39E−07 | 1.02E−04 | 1.41E−08 | 1.60E−29 | 0 |
| OXYGEN | 1.12E−05 | 1.12E−05 | 1.11E−05 | 1.74E−03 | 2.87E−06 | 3.20E−28 | 0 |
| CARBON- | 1.04E−04 | 1.04E−04 | 1.03E−04 | 0.01652 | 4.44E−06 | 2.38E−25 | 0 |
| MONOXIDE | | | | | | | |
| CARBON- | 7.63E−04 | 7.63E−04 | 7.61E−04 | 0.109297 | 8.15E−04 | 2.18E−20 | 0 |
| DIOXIDE | | | | | | | |
| METHANE | 2.11E−04 | 2.11E−04 | 2.10E−04 | 0.033384 | 2.26E−05 | 6.36E−24 | 0 |
| ACETYLENE | 6.64E−04 | 6.64E−04 | 6.62E−04 | 0.052361 | 3.43E−03 | 1.09E−15 | 0 |
| ETHYLENE | 4.75E−03 | 4.75E−03 | 4.74E−03 | 0.715472 | 2.87E−03 | 1.28E−19 | 0 |
| ETHANE | 5.07E−05 | 5.07E−05 | 5.05E−05 | 7.77E−03 | 2.16E−05 | 2.70E−22 | 0 |
| PROPADIENE | 1.95E−05 | 1.95E−05 | 1.94E−05 | 1.83E−03 | 8.21E−05 | 2.87E−19 | 0 |
| METHYL- | 1.37E−07 | 1.37E−07 | 1.37E−07 | 1.42E−05 | 4.90E−07 | 2.93E−22 | 0 |
| ACETYLENE | | | | | | | |
| PROPYLENE | 1.06E−04 | 1.06E−04 | 1.06E−04 | 1.46E−02 | 1.49E−04 | 6.70E−21 | 0 |
| PROPANE | 1.46E−06 | 1.46E−06 | 1.46E−06 | 2.13E−04 | 1.30E−06 | 6.21E−24 | 0 |
| 1,3-BUTADIENE | 2.98E−04 | 2.98E−04 | 2.97E−04 | 0.020207 | 1.75E−03 | 7.41E−17 | 0 |
| ISOBUTYLENE | 7.46E−06 | 7.46E−06 | 7.45E−06 | 8.50E−04 | 2.19E−05 | 1.72E−20 | 0 |
| CIS-2-BUTENE | 5.62E−06 | 5.62E−06 | 5.61E−06 | 6.60E−04 | 1.52E−05 | 1.88E−20 | 0 |
| TRANS-2-BUTENE | 4.44E−06 | 4.44E−06 | 4.42E−06 | 4.47E−04 | 1.67E−05 | 8.63E−16 | 0 |
| 1-HEXENE | 1.23E−04 | 1.23E−04 | 1.23E−04 | 4.25E−03 | 9.83E−04 | 9.88E−16 | 0 |
| N-HEXANE | 2.29E−04 | 2.29E−04 | 2.29E−04 | 0.015597 | 1.34E−03 | 1.60E−17 | 0 |
| 1-HEXADECENE | 2.71E−04 | 2.71E−04 | 2.70E−04 | 1.11E−07 | 1.32E−04 | 2.88E−04 | 0 |
| N-METHYL-2- | 0.992367 | 0.992367 | 0.992387 | 2.73E−03 | 0.988347 | 0.999713 | 1 |
| PYRROLIDONE | | | | | | | |

| Stream (Example 2) | 100 | 101 | 102 | 103 | 104 | 107 | 109 | 108 |
|---|---|---|---|---|---|---|---|---|
| Mass Flow lb/hr | | | | | | | | |
| HYDROGEN | 7.721192 | 6.176954 | 1.544238 | 6.150857 | 0.026471 | 1.57071 | 0.657217 | 0.036248 |
| NITROGEN | 2.207047 | 1.765637 | 0.441409 | 1.757588 | 8.32E−03 | 0.449731 | 0.426595 | 0.023135 |
| OXYGEN | 0.517188 | 0.41375 | 0.103438 | 0.322376 | 0.096786 | 0.200224 | 0.175033 | 0.025191 |
| CARBON- | 75.73806 | 60.59045 | 15.14761 | 60.08296 | 0.551513 | 15.69913 | 14.22233 | 1.476791 |
| MONOXIDE | | | | | | | | |
| CARBON- | 4.428842 | 3.543074 | 0.885768 | 3.081917 | 0.970227 | 1.855996 | 0.986645 | 0.86935 |
| DIOXIDE | | | | | | | | |
| METHANE | 16.74716 | 13.39772 | 3.349431 | 13.1218 | 0.327205 | 3.676636 | 3.104139 | 0.572496 |
| ACETYLENE | 11.21 | 8.967999 | 2.242 | 0.099857 | 9.130086 | 11.37209 | 0.053634 | 0.287074 |
| ETHYLENE | 1.00345 | 0.80276 | 0.20069 | 2.710475 | 0.113408 | 0.314098 | 7.03754 | 4.793932 |
| ETHANE | 0 | 0 | 0 | 0.0231 | 1.70E−16 | 1.70E−16 | 0.139923 | 0.070205 |
| PROPADIENE | 0.207169 | 0.165735 | 0.041434 | 7.17E−03 | 0.17039 | 0.211824 | 3.49E−03 | 0.013452 |
| METHYL- | 1.70E−03 | 1.36E−03 | 3.40E−04 | 7.16E−05 | 1.37E−03 | 1.71E−03 | 3.99E−05 | 9.72E−05 |
| ACETYLENE | | | | | | | | |
| PROPYLENE | 0 | 0 | 0 | 0.067548 | 4.27E−08 | 4.27E−08 | 0.098761 | 0.103093 |
| PROPANE | 0 | 0 | 0 | 9.76E−04 | 5.06E−13 | 5.06E−13 | 2.85E−03 | 1.85E−03 |
| 1,3-BUTADIENE | 0 | 0 | 0 | 0.075168 | 0.169045 | 0.169045 | 0.046431 | 0.264361 |
| ISOBUTYLENE | 0 | 0 | 0 | 6.06E−03 | 2.94E−04 | 2.94E−04 | 3.58E−03 | 7.74E−03 |
| CIS-2-BUTENE | 0 | 0 | 0 | 4.70E−03 | 7.95E−05 | 7.95E−05 | 2.72E−03 | 5.93E−03 |
| TRANS-2-BUTENE | 0 | 0 | 0 | 2.97E−03 | 8.06E−04 | 8.06E−04 | 5.77E−04 | 4.39E−03 |
| 1-HEXENE | 0.218195 | 0.174556 | 0.043639 | 0.017832 | 0.313781 | 0.35742 | 0.014246 | 0.164464 |
| N-HEXANE | 0 | 0 | 0 | 0.091784 | 0.208033 | 0.208033 | 0.06589 | 0.325134 |
| 1-HEXADECENE | 0 | 0 | 0 | 1.41E−05 | 0.920409 | 0.920409 | 1.78E−04 | 0.921456 |
| N-METHYL-2- | 0 | 0 | 0 | 0.091901 | 1495.275 | 1495.275 | 0.525966 | 1494.749 |
| PYRROLIDONE | | | | | | | | |
| Mass Frac | | | | | | | | |
| HYDROGEN | 0.064343 | 0.064343 | 0.064343 | 0.070122 | 1.76E−05 | 1.03E−03 | 0.02384 | 2.41E−05 |
| NITROGEN | 0.018392 | 0.018392 | 0.018392 | 0.020037 | 5.52E−06 | 2.94E−04 | 0.015474 | 1.54E−05 |
| OXYGEN | 4.31E−03 | 4.31E−03 | 4.31E−03 | 3.68E−03 | 6.42E−05 | 1.31E−04 | 6.35E−03 | 1.67E−05 |
| CARBON- | 0.631151 | 0.631151 | 0.631151 | 0.684963 | 3.66E−04 | 0.010246 | 0.515904 | 9.81E−04 |
| MONOXIDE | | | | | | | | |
| CARBON- | 0.036907 | 0.036907 | 0.036907 | 0.035135 | 6.43E−04 | 1.21E−03 | 0.03579 | 5.78E−04 |
| DIOXIDE | | | | | | | | |
| METHANE | 0.13956 | 0.13956 | 0.13956 | 0.149592 | 2.17E−04 | 2.40E−03 | 0.1126 | 3.80E−04 |
| ACETYLENE | 0.093417 | 0.093417 | 0.093417 | 1.14E−03 | 6.05E−03 | 7.42E−03 | 1.95E−03 | 1.91E−04 |
| ETHYLENE | 8.36E−03 | 8.36E−03 | 8.36E−03 | 0.0309 | 7.52E−05 | 2.05E−04 | 0.255281 | 3.19E−03 |
| ETHANE | 0 | 0 | 0 | 2.63E−04 | 1.13E−19 | 1.11E−19 | 5.08E−03 | 4.67E−05 |
| PROPADIENE | 1.73E−03 | 1.73E−03 | 1.73E−03 | 8.18E−05 | 1.13E−04 | 1.38E−04 | 1.27E−04 | 8.94E−06 |
| METHYL- | 1.42E−05 | 1.42E−05 | 1.42E−05 | 8.16E−07 | 9.10E−07 | 1.12E−06 | 1.45E−06 | 6.46E−08 |
| ACETYLENE | | | | | | | | |
| PROPYLENE | 0 | 0 | 0 | 7.70E−04 | 2.83E−11 | 2.79E−11 | 3.58E−03 | 6.85E−05 |
| PROPANE | 0 | 0 | 0 | 1.11E−05 | 3.35E−16 | 3.30E−16 | 1.03E−04 | 1.23E−06 |
| 1,3-BUTADIENE | 0 | 0 | 0 | 8.57E−04 | 1.12E−04 | 1.10E−04 | 1.68E−03 | 1.76E−04 |
| ISOBUTYLENE | 0 | 0 | 0 | 6.90E−05 | 1.95E−07 | 1.92E−07 | 1.30E−04 | 5.14E−06 |
| CIS-2-BUTENE | 0 | 0 | 0 | 5.36E−05 | 5.27E−08 | 5.19E−08 | 9.88E−05 | 3.94E−06 |
| TRANS-2-BUTENE | 0 | 0 | 0 | 3.38E−05 | 5.34E−07 | 5.26E−07 | 2.09E−05 | 2.92E−06 |
| 1-HEXENE | 1.82E−03 | 1.82E−03 | 1.82E−03 | 2.03E−04 | 2.08E−04 | 2.33E−04 | 5.17E−04 | 1.09E−04 |
| N-HEXANE | 0 | 0 | 0 | 1.05E−03 | 1.38E−04 | 1.36E−04 | 2.39E−03 | 2.16E−04 |
| 1-HEXADECENE | 0 | 0 | 0 | 1.61E−07 | 6.10E−04 | 6.01E−04 | 6.47E−06 | 6.12E−04 |
| N-METHYL-2- | 0 | 0 | 0 | 1.05E−03 | 0.991376 | 0.975848 | 0.019079 | 0.993377 |
| PYRROLIDONE | | | | | | | | |
| Total Flow lbmol/hr | 8.247701 | 6.59816 | 1.64954 | 6.263862 | 15.53453 | 17.18407 | 1.337674 | 15.40815 |
| Total Flow lb/hr | 120 | 96 | 24 | 87.71712 | 1508.283 | 1532.283 | 27.5678 | 1504.715 |
| Total Flow cuft/hr | 164.2334 | 131.3867 | 32.84669 | 270.2179 | 31.34932 | 115.7638 | 40.03559 | 32.75526 |
| Temperature F. | 85 | 85 | 85 | 99.83898 | 103.7181 | 187.6713 | 229.8431 | 229.8431 |
| Pressure psia | 294.6959 | 294.6959 | 294.6959 | 139.6959 | 141.6959 | 140.6959 | 247.6959 | 247.6959 |
| Density lb/cuft | 0.730667 | 0.730667 | 0.730667 | 0.324616 | 48.11215 | 13.23629 | 0.688583 | 45.93812 |
| Average MW | 14.54951 | 14.54951 | 14.54951 | 14.00368 | 97.09227 | 89.16879 | 20.60877 | 97.6571 |

| Stream (Example 2) | 114 | 112 | 113 | 117 | 116 | 121 | 122 |
|---|---|---|---|---|---|---|---|
| Mass Flow lb/hr | | | | | | | |
| HYDROGEN | 0.017011 | 0.019237 | 0.676454 | 0.676453 | 5.27E−07 | 0.016628 | 0.693081 |
| NITROGEN | 0.010746 | 0.01239 | 0.438985 | 0.438984 | 4.12E−07 | 1.05E−02 | 0.449452 |
| OXYGEN | 0.016573 | 8.62E−03 | 0.183651 | 0.183648 | 2.89E−06 | 1.10E−02 | 0.194673 |
| CARBON- | 0.923045 | 0.553746 | 14.77608 | 14.77605 | 2.68E−05 | 0.877921 | 15.65397 |
| MONOXIDE | | | | | | | |
| CARBON- | 0.812216 | 0.057134 | 1.04378 | 1.043698 | 8.16E−05 | 0.290186 | 1.333884 |
| DIOXIDE | | | | | | | |
| METHANE | 0.428117 | 0.14438 | 3.248519 | 3.248502 | 1.72E−05 | 0.375542 | 3.624044 |
| ACETYLENE | 0.283788 | 3.29E−03 | 0.056919 | 0.05687 | 4.90E−05 | 1.52E−02 | 0.072047 |
| ETHYLENE | 4.395977 | 0.397955 | 7.435495 | 7.435226 | 2.69E−04 | 2.3233 | 9.758526 |
| ETHANE | 0.062514 | 7.69E−03 | 0.147614 | 0.14761 | 3.62E−06 | 3.88E−02 | 0.186435 |
| PROPADIENE | 0.013241 | 2.11E−04 | 3.71E−03 | 3.70E−03 | 1.78E−06 | 1.11E−03 | 4.81E−03 |
| METHYL- | 9.48E−05 | 2.38E−06 | 4.22E−05 | 4.22E−05 | 1.78E−08 | 9.53E−06 | 5.18E−05 |
| ACETYLENE | | | | | | | |
| PROPYLENE | 0.097373 | 5.72E−03 | 0.104481 | 0.10447 | 1.14E−05 | 2.81E−02 | 0.132574 |
| PROPANE | 1.69E−03 | 1.60E−04 | 3.01E−03 | 3.01E−03 | 1.92E−07 | 6.88E−04 | 3.70E−03 |
| 1,3-BUTADIENE | 0.26156 | 2.80E−03 | 0.049233 | 0.04918 | 5.24E−05 | 1.11E−02 | 0.060319 |
| ISOBUTYLENE | 7.52E−03 | 2.13E−04 | 3.80E−03 | 3.80E−03 | 1.12E−06 | 1.01E−03 | 4.81E−03 |
| CIS-2-BUTENE | 5.77E−03 | 1.65E−04 | 2.89E−03 | 2.89E−03 | 7.44E−07 | 8.64E−04 | 3.75E−03 |
| TRANS-2-BUTENE | 4.36E−03 | 3.51E−05 | 6.12E−04 | 6.12E−04 | 1.89E−07 | 4.89E−04 | 1.10E−03 |
| 1-HEXENE | 0.163599 | 8.65E−04 | 0.015111 | 0.015063 | 4.73E−05 | 2.56E−03 | 0.017626 |
| N-HEXANE | 0.321094 | 4.04E−03 | 0.06993 | 0.069853 | 7.69E−05 | 1.37E−02 | 0.083518 |
| 1-HEXADECENE | 0.921445 | 1.15E−05 | 1.90E−04 | 4.72E−06 | 1.85E−04 | 1.92E−06 | 6.64E−06 |
| N-METHYL-2- | 1494.717 | 0.031419 | 0.557385 | 0.053761 | 0.503624 | 1.11E−02 | 0.064896 |
| PYRROLIDONE | | | | | | | |
| Mass Frac | | | | | | | |
| HYDROGEN | 1.13E−05 | 0.015388 | 0.023473 | 0.023892 | 1.04E−06 | 4.13E−03 | 0.021429 |
| NITROGEN | 7.15E−06 | 9.91E−03 | 0.015233 | 0.015504 | 8.16E−07 | 2.60E−03 | 0.013896 |
| OXYGEN | 1.10E−05 | 6.89E−03 | 6.37E−03 | 6.49E−03 | 5.73E−06 | 2.74E−03 | 6.02E−03 |
| CARBON- | 6.14E−04 | 0.442969 | 0.51274 | 0.521874 | 5.31E−05 | 0.217855 | 0.483995 |
| MONOXIDE | | | | | | | |
| CARBON- | 5.40E−04 | 0.045705 | 0.03622 | 0.036862 | 1.62E−04 | 0.072009 | 0.041241 |
| DIOXIDE | | | | | | | |
| METHANE | 2.85E−04 | 0.115496 | 0.112726 | 0.114734 | 3.41E−05 | 0.09319 | 0.112049 |
| ACETYLENE | 1.89E−04 | 2.63E−03 | 1.98E−03 | 2.01E−03 | 9.72E−05 | 3.77E−03 | 2.23E−03 |
| ETHYLENE | 2.92E−03 | 0.318344 | 0.258017 | 0.262604 | 5.33E−04 | 0.576523 | 0.301717 |
| ETHANE | 4.16E−05 | 6.15E−03 | 5.12E−03 | 5.21E−03 | 7.17E−06 | 9.63E−03 | 5.76E−03 |
| PROPADIENE | 8.81E−06 | 1.69E−04 | 1.29E−04 | 1.31E−04 | 3.53E−06 | 2.75E−04 | 1.49E−04 |
| METHYL- | 6.31E−08 | 1.90E−06 | 1.47E−06 | 1.49E−06 | 3.53E−08 | 2.37E−06 | 1.60E−06 |
| ACETYLENE | | | | | | | |
| PROPYLENE | 6.48E−05 | 4.58E−03 | 3.63E−03 | 3.69E−03 | 2.26E−05 | 6.97E−03 | 4.10E−03 |
| PROPANE | 1.12E−06 | 1.28E−04 | 1.05E−04 | 1.06E−04 | 3.81E−07 | 1.71E−04 | 1.14E−04 |
| 1,3-BUTADIENE | 1.74E−04 | 2.24E−03 | 1.71E−03 | 1.74E−03 | 1.04E−04 | 2.76E−03 | 1.86E−03 |
| ISOBUTYLENE | 5.00E−06 | 1.71E−04 | 1.32E−04 | 1.34E−04 | 2.21E−06 | 2.51E−04 | 1.49E−04 |
| CIS-2-BUTENE | 3.84E−06 | 1.32E−04 | 1.00E−04 | 1.02E−04 | 1.48E−06 | 2.14E−04 | 1.16E−04 |
| TRANS-2-BUTENE | 2.90E−06 | 2.81E−05 | 2.12E−05 | 2.16E−05 | 3.76E−07 | 1.21E−04 | 3.41E−05 |
| 1-HEXENE | 1.09E−04 | 6.92E−04 | 5.24E−04 | 5.32E−04 | 9.38E−05 | 6.36E−04 | 5.45E−04 |
| N-HEXANE | 2.14E−04 | 3.23E−03 | 2.43E−03 | 2.47E−03 | 1.52E−04 | 3.39E−03 | 2.58E−03 |
| 1-HEXADECENE | 6.13E−04 | 9.22E−06 | 6.59E−06 | 1.67E−07 | 3.67E−04 | 4.76E−07 | 2.05E−07 |
| N-METHYL-2- | 0.994182 | 0.025133 | 0.019342 | 1.90E−03 | 0.998358 | 2.76E−03 | 2.01E−03 |
| PYRROLIDONE | | | | | | | |
| Total Flow lbmol/hr | 15.35268 | 0.055467 | 1.393141 | 1.388041 | 5.10E−03 | 0.156263 | 1.544303 |
| Total Flow lb/hr | 1503.465 | 1.25008 | 28.81788 | 28.31343 | 0.504453 | 4.029849 | 32.34328 |
| Total Flow cuft/hr | 32.73058 | 2.490758 | 62.49332 | 406.0454 | 0.01037 | 46.86407 | 453.1131 |
| Temperature F. | 230.3602 | 230.3602 | 228.082 | 77.75407 | 77.75407 | 92.7253 | 79.50541 |
| Pressure psia | 164.6959 | 164.6959 | 164.6959 | 19.69595 | 19.69595 | 19.69595 | 19.69595 |
| Density lb/cuft | 45.93457 | 0.501887 | 0.461136 | 0.06973 | 48.64507 | 0.08599 | 0.07138 |
| Average MW | 97.9285 | 22.53738 | 20.68555 | 20.39812 | 98.91698 | 25.78897 | 20.9436 |

| Stream (Example 2) | 123 | 126 | 124 | 127 | 128 | 129 | 118 |
|---|---|---|---|---|---|---|---|
| Mass Flow lb/hr | | | | | | | |
| HYDROGEN | 3.84E−04 | 9.59E−06 | 3.74E−04 | 9.57E−06 | 1.89E−08 | 3.21E−28 | 0 |
| NITROGEN | 2.79E−04 | 6.98E−06 | 2.72E−04 | 6.96E−06 | 1.51E−08 | 1.56E−28 | 0 |
| OXYGEN | 5.55E−03 | 1.39E−04 | 5.41E−03 | 1.35E−04 | 3.51E−06 | 3.56E−27 | 0 |
| CARBON- | 0.04515 | 1.13E−03 | 0.044022 | 1.12E−03 | 4.75E−06 | 2.32E−24 | 0 |
| MONOXIDE | | | | | | | |
| CARBON- | 0.522123 | 0.013053 | 0.50907 | 0.011684 | 1.37E−03 | 3.34E−19 | 0 |
| DIOXIDE | | | | | | | |
| METHANE | 0.052592 | 1.31E−03 | 0.051277 | 1.30E−03 | 1.38E−05 | 3.55E−23 | 0 |
| ACETYLENE | 0.268661 | 6.72E−03 | 0.261944 | 3.31E−03 | 3.40E−03 | 9.90E−15 | 0 |
| ETHYLENE | 2.072947 | 0.051824 | 2.021123 | 0.048756 | 3.07E−03 | 1.25E−18 | 0 |
| ETHANE | 0.023693 | 5.92E−04 | 0.0231 | 5.68E−04 | 2.48E−05 | 2.83E−21 | 0 |
| PROPADIENE | 0.012132 | 3.03E−04 | 0.011829 | 1.78E−04 | 1.26E−04 | 4.00E−18 | 0 |
| METHYL- | 8.53E−05 | 2.13E−06 | 8.32E−05 | 1.38E−06 | 7.49E−07 | 4.09E−21 | 0 |
| ACETYLENE | | | | | | | |
| PROPYLENE | 0.06928 | 1.73E−03 | 0.067548 | 1.49E−03 | 2.39E−04 | 9.81E−20 | 0 |
| PROPANE | 1.00E−03 | 2.50E−05 | 9.76E−04 | 2.29E−05 | 2.18E−06 | 9.54E−23 | 0 |
| 1,3-BUTADIENE | 0.250475 | 6.26E−03 | 0.244213 | 2.66E−03 | 3.61E−03 | 1.39E−15 | 0 |
| ISOBUTYLENE | 6.51E−03 | 1.63E−04 | 6.35E−03 | 1.16E−04 | 4.69E−05 | 3.37E−19 | 0 |
| CIS-2-BUTENE | 4.90E−03 | 1.23E−04 | 4.78E−03 | 9.00E−05 | 3.27E−05 | 3.67E−19 | 0 |
| TRANS-2-BUTENE | 3.87E−03 | 9.68E−05 | 3.77E−03 | 6.09E−05 | 3.58E−05 | 1.69E−14 | 0 |
| 1-HEXENE | 0.161084 | 4.03E−03 | 0.157057 | 8.68E−04 | 3.16E−03 | 2.90E−14 | 0 |
| N-HEXANE | 0.307505 | 7.69E−03 | 0.299817 | 3.26E−03 | 4.42E−03 | 4.81E−16 | 0 |
| 1-HEXADECENE | 0.944024 | 2.36E−02 | 0.920423 | 6.04E−08 | 1.13E−03 | 0.022467 | 0 |
| N-METHYL-2- | 1529.713 | 38.24283 | 1495.367 | 6.57E−04 | 3.739166 | 34.503 | 3.896352 |
| PYRROLIDONE | | | | | | | |
| Mass Frac | | | | | | | |
| HYDROGEN | 2.50E−07 | 2.50E−07 | 2.49E−07 | 1.25E−04 | 5.01E−09 | 9.31E−30 | 0 |
| NITROGEN | 1.82E−07 | 1.82E−07 | 1.81E−07 | 9.13E−05 | 4.02E−09 | 4.51E−30 | 0 |
| OXYGEN | 3.62E−06 | 3.62E−06 | 3.61E−06 | 1.77E−03 | 9.32E−07 | 1.03E−28 | 0 |
| CARBON- | 2.94E−05 | 2.94E−05 | 2.93E−05 | 0.014731 | 1.26E−06 | 6.73E−26 | 0 |
| MONOXIDE | | | | | | | |
| CARBON- | 3.40E−04 | 3.40E−04 | 3.39E−04 | 0.153129 | 3.64E−04 | 9.67E−21 | 0 |
| DIOXIDE | | | | | | | |
| METHANE | 3.43E−05 | 3.43E−05 | 3.42E−05 | 0.01705 | 3.68E−06 | 1.03E−24 | 0 |
| ACETYLENE | 1.75E−04 | 1.75E−04 | 1.75E−04 | 0.043402 | 9.06E−04 | 2.87E−16 | 0 |
| ETHYLENE | 1.35E−03 | 1.35E−03 | 1.35E−03 | 0.638973 | 8.16E−04 | 3.62E−20 | 0 |
| ETHANE | 1.54E−05 | 1.54E−05 | 1.54E−05 | 7.44E−03 | 6.58E−06 | 8.19E−23 | 0 |
| PROPADIENE | 7.91E−06 | 7.91E−06 | 7.89E−06 | 2.33E−03 | 3.34E−05 | 1.16E−19 | 0 |
| METHYL- | 5.56E−08 | 5.56E−08 | 5.55E−08 | 1.81E−05 | 1.99E−07 | 1.19E−22 | 0 |
| ACETYLENE | | | | | | | |
| PROPYLENE | 4.51E−05 | 4.51E−05 | 4.50E−05 | 0.019568 | 6.35E−05 | 2.84E−21 | 0 |
| PROPANE | 6.53E−07 | 6.53E−07 | 6.51E−07 | 3.00E−04 | 5.80E−07 | 2.76E−24 | 0 |
| 1,3-BUTADIENE | 1.63E−04 | 1.63E−04 | 1.63E−04 | 0.034796 | 9.59E−04 | 4.04E−17 | 0 |
| ISOBUTYLENE | 4.24E−06 | 4.24E−06 | 4.23E−06 | 1.52E−03 | 1.25E−05 | 9.75E−21 | 0 |
| CIS-2-BUTENE | 3.20E−06 | 3.20E−06 | 3.19E−06 | 1.18E−03 | 8.68E−06 | 1.06E−20 | 0 |
| TRANS-2-BUTENE | 2.52E−06 | 2.52E−06 | 2.52E−06 | 7.98E−04 | 9.53E−06 | 4.88E−16 | 0 |
| 1-HEXENE | 1.05E−04 | 1.05E−04 | 1.05E−04 | 1.14E−02 | 8.40E−04 | 8.39E−16 | 0 |
| N-HEXANE | 2.00E−04 | 2.00E−04 | 2.00E−04 | 0.042789 | 1.18E−03 | 1.39E−17 | 0 |
| 1-HEXADECENE | 6.15E−04 | 6.15E−04 | 6.14E−04 | 7.91E−07 | 3.02E−04 | 6.51E−04 | 0 |
| N-METHYL-2- | 0.996903 | 0.996903 | 0.996911 | 8.61E−03 | 0.994496 | 0.999349 | 1 |
| PYRROLIDONE | | | | | | | |
| Total Flow lbmol/hr | 15.54967 | 0.388742 | 15.20023 | 2.43E−03 | 0.038164 | 0.348149 | 0.039304 |
| Total Flow lb/hr | 1534.465 | 38.36163 | 1500 | 0.076304 | 3.75986 | 34.52547 | 3.896352 |
| Total Flow cuft/hr | 31.71363 | 0.792841 | 31.02751 | 1.531042 | 0.078198 | 0.802971 | 0.080227 |
| Temperature F. | 92.7253 | 92.72516 | 98.9772 | 110 | 110 | 371.0184 | 85 |
| Pressure psia | 19.69595 | 19.69595 | 304.6959 | 9.668387 | 9.668387 | 10.05512 | 29.69595 |
| Density lb/cuft | 48.38505 | 48.38505 | 48.34419 | 0.049838 | 48.08126 | 42.99719 | 48.56632 |
| Average MW | 98.68153 | 98.68153 | 98.6827 | 31.4124 | 98.51966 | 99.16863 | 99.1326 |

TABLE 3

| Stream (Example 3) | 200 | 201 | 202 | 203 | 204 | 207 | 209 | 208 |
|---|---|---|---|---|---|---|---|---|
| Substream: MIXED | | | | | | | | |
| Mole Flow lbmol/hr | | | | | | | | |
| HYDROGEN | 3.830185 | 3.064148 | 0.766037 | 3.052067 | 0.01208 | 0.778117 | 0.325169 | 0.017574 |
| NITROGEN | 0.078785 | 0.063028 | 0.015757 | 0.062739 | 2.89E−04 | 0.016046 | 0.015226 | 8.20E−04 |
| OXYGEN | 0.016163 | 0.01293 | 3.23E−03 | 8.47E−03 | 4.46E−03 | 7.70E−03 | 6.73E−03 | 9.65E−04 |
| CARBON- | 2.703926 | 2.163141 | 0.540785 | 2.144063 | 0.019078 | 0.559864 | 0.507623 | 0.05224 |
| MONOXIDE | | | | | | | | |
| CARBON- | 0.100633 | 0.080506 | 0.020127 | 0.054946 | 0.02556 | 0.045687 | 0.024275 | 0.021412 |
| DIOXIDE | | | | | | | | |
| METHANE | 1.043907 | 0.835126 | 0.208782 | 0.814179 | 0.020947 | 0.229728 | 0.193939 | 0.035789 |
| ACETYLENE | 0.430527 | 0.344421 | 0.086105 | 2.82E−03 | 0.351139 | 0.437244 | 2.06E−03 | 0.011037 |
| ETHYLENE | 0.035769 | 0.028615 | 7.15E−03 | 0.0242 | 4.41E−03 | 0.011569 | 0.251304 | 0.171274 |
| ETHANE | 0 | 0 | 0 | 0 | 0 | 0 | 4.66E−03 | 2.34E−03 |
| PROPADIENE | 5.17E−03 | 4.14E−03 | 1.03E−03 | 1.63E−05 | 4.15E−03 | 5.19E−03 | 8.56E−05 | 3.29E−04 |
| METHYL- | 4.25E−05 | 3.40E−05 | 8.50E−06 | 1.63E−08 | 3.40E−05 | 4.25E−05 | 9.87E−07 | 2.41E−06 |
| ACETYLENE | | | | | | | | |
| PROPYLENE | 0 | 0 | 0 | 1.20E−11 | 6.54E−17 | 6.54E−17 | 2.30E−03 | 2.40E−03 |
| PROPANE | 0 | 0 | 0 | 0 | 0 | 0 | 6.34E−05 | 4.11E−05 |
| 1,3-BUTADIENE | 0 | 0 | 0 | 1.04E−03 | 3.21E−03 | 3.21E−03 | 8.72E−04 | 4.96E−03 |
| ISOBUTYLENE | 0 | 0 | 0 | 7.46E−09 | 1.31E−09 | 1.31E−09 | 6.23E−05 | 1.34E−04 |
| CIS-2-BUTENE | 0 | 0 | 0 | 7.34E−09 | 6.08E−10 | 6.08E−10 | 4.82E−05 | 1.05E−04 |
| TRANS-2-BUTENE | 0 | 0 | 0 | 3.92E−05 | 1.31E−05 | 1.31E−05 | 1.02E−05 | 7.73E−05 |
| 1-HEXENE | 2.59E−03 | 2.07E−03 | 5.19E−04 | 1.22E−04 | 3.37E−03 | 3.88E−03 | 1.55E−04 | 1.79E−03 |
| N-HEXANE | 0 | 0 | 0 | 7.53E−04 | 2.43E−03 | 2.43E−03 | 7.38E−04 | 3.63E−03 |
| 1-HEXADECENE | 0 | 0 | 0 | 7.32E−10 | 9.39E−05 | 9.39E−05 | 1.93E−08 | 9.94E−05 |
| N-METHYL-2- | 0 | 0 | 0 | 5.31E−04 | 15.12168 | 15.12168 | 5.31E−03 | 15.11636 |
| PYRROLIDONE | | | | | | | | |
| Mole Frac | | | | | | | | |
| HYDROGEN | 0.464394 | 0.464394 | 0.464394 | 0.494985 | 7.76E−04 | 0.04518 | 0.242548 | 1.14E−03 |
| NITROGEN | 9.55E−03 | 9.55E−03 | 9.55E−03 | 0.010175 | 1.86E−05 | 9.32E−04 | 0.011357 | 5.31E−05 |
| OXYGEN | 1.96E−03 | 1.96E−03 | 1.96E−03 | 1.37E−03 | 2.87E−04 | 4.47E−04 | 5.02E−03 | 6.25E−05 |
| CARBON- | 0.32784 | 0.32784 | 0.32784 | 0.347725 | 1.23E−03 | 0.032508 | 0.378643 | 3.38E−03 |
| MONOXIDE | | | | | | | | |
| CARBON- | 0.012201 | 0.012201 | 0.012201 | 8.91E−03 | 1.64E−03 | 2.65E−03 | 0.018107 | 1.39E−03 |
| DIOXIDE | | | | | | | | |
| METHANE | 0.12657 | 0.12657 | 0.12657 | 0.132044 | 1.35E−03 | 0.013339 | 0.144662 | 2.32E−03 |
| ACETYLENE | 0.0522 | 0.0522 | 0.0522 | 4.57E−04 | 0.022548 | 0.025388 | 1.54E−03 | 7.15E−04 |
| ETHYLENE | 4.34E−03 | 4.34E−03 | 4.34E−03 | 3.92E−03 | 2.84E−04 | 6.72E−04 | 0.187451 | 0.01109 |
| ETHANE | 0 | 0 | 0 | 0 | 0 | 0 | 3.47E−03 | 1.51E−04 |
| PROPADIENE | 6.27E−04 | 6.27E−04 | 6.27E−04 | 2.65E−06 | 2.67E−04 | 3.01E−04 | 6.38E−05 | 2.13E−05 |
| METHYL- | 5.15E−06 | 5.15E−06 | 5.15E−06 | 2.64E−09 | 2.18E−06 | 2.47E−06 | 7.36E−07 | 1.56E−07 |
| ACETYLENE | | | | | | | | |
| PROPYLENE | 0 | 0 | 0 | 1.94E−12 | 4.20E−18 | 3.80E−18 | 1.72E−03 | 1.56E−04 |
| PROPANE | 0 | 0 | 0 | 0 | 0 | 0 | 4.73E−05 | 2.66E−06 |
| 1,3-BUTADIENE | 0 | 0 | 0 | 1.68E−04 | 2.06E−04 | 1.86E−04 | 6.50E−04 | 3.21E−04 |
| ISOBUTYLENE | 0 | 0 | 0 | 1.21E−09 | 8.41E−11 | 7.60E−11 | 4.65E−05 | 8.70E−06 |
| CIS-2-BUTENE | 0 | 0 | 0 | 1.19E−09 | 3.91E−11 | 3.53E−11 | 3.60E−05 | 6.79E−06 |
| TRANS-2-BUTENE | 0 | 0 | 0 | 6.35E−06 | 8.43E−07 | 7.63E−07 | 7.59E−06 | 5.00E−06 |
| 1-HEXENE | 3.14E−04 | 3.14E−04 | 3.14E−04 | 1.97E−05 | 2.16E−04 | 2.26E−04 | 1.16E−04 | 1.16E−04 |
| N-HEXANE | 0 | 0 | 0 | 1.22E−04 | 1.56E−04 | 1.41E−04 | 5.51E−04 | 2.35E−04 |
| 1-HEXADECENE | 0 | 0 | 0 | 1.19E−10 | 6.03E−06 | 5.45E−06 | 1.44E−08 | 6.43E−06 |
| N-METHYL-2- | 0 | 0 | 0 | 8.61E−05 | 0.971022 | 0.878019 | 3.96E−03 | 0.978824 |
| PYRROLIDONE | | | | | | | | |

| Stream (Example 3) | 214 | 212 | 213 | 222 | 216 | 221 | 223 |
|---|---|---|---|---|---|---|---|
| Substream: MIXED | | | | | | | |
| Mole Flow lbmol/hr | | | | | | | |
| HYDROGEN | 8.18E−03 | 9.39E−03 | 0.334561 | 0.342744 | 6.87E−04 | 8.87E−03 | 2.01E−25 |
| NITROGEN | 3.81E−04 | 4.39E−04 | 0.015666 | 0.016046 | 3.72E−05 | 4.18E−04 | 1.13E−27 |
| OXYGEN | 6.36E−04 | 3.30E−04 | 7.06E−03 | 7.70E−03 | 2.88E−03 | 3.51E−03 | 2.43E−19 |
| CARBON- | 0.032612 | 0.019629 | 0.527252 | 0.559864 | 2.49E−03 | 0.035101 | 3.87E−22 |
| MONOXIDE | | | | | | | |
| CARBON- | 0.020014 | 1.40E−03 | 0.025673 | 0.045687 | 8.01E−03 | 0.028027 | 4.60E−12 |
| DIOXIDE | | | | | | | |
| METHANE | 0.026807 | 8.98E−03 | 0.202921 | 0.229728 | 2.86E−03 | 0.029671 | 1.43E−19 |
| ACETYLENE | 0.010912 | 1.26E−04 | 2.19E−03 | 4.99E−07 | 0.13932 | 0.137132 | 0.0131 |
| ETHYLENE | 0.157139 | 0.014135 | 0.265439 | 0.422578 | 0.034646 | 0.191785 | 3.31E−12 |
| ETHANE | 2.08E−03 | 2.55E−04 | 4.91E−03 | 7.00E−03 | 3.92E−04 | 2.48E−03 | 1.67E−15 |
| PROPADIENE | 3.24E−04 | 5.14E−06 | 9.07E−05 | 3.71E−04 | 6.42E−03 | 6.70E−03 | 4.34E−05 |
| METHYL- | 2.35E−06 | 5.85E−08 | 1.05E−06 | 3.40E−06 | 1.36E−05 | 1.59E−05 | 1.72E−09 |
| ACETYLENE | | | | | | | |
| PROPYLENE | 2.27E−03 | 1.33E−04 | 2.43E−03 | 4.71E−03 | 1.25E−03 | 3.52E−03 | 1.64E−11 |
| PROPANE | 3.76E−05 | 3.54E−06 | 6.70E−05 | 1.05E−04 | 1.53E−05 | 5.29E−05 | 1.56E−15 |
| 1,3-BUTADIENE | 4.91E−03 | 5.23E−05 | 9.24E−04 | 2.03E−10 | 1.99E−03 | 1.07E−03 | 5.83E−03 |
| ISOBUTYLENE | 1.31E−04 | 3.69E−06 | 6.60E−05 | 1.97E−04 | 2.38E−04 | 3.68E−04 | 1.20E−08 |
| CIS-2-BUTENE | 1.02E−04 | 2.91E−06 | 5.12E−05 | 1.53E−04 | 1.47E−04 | 2.49E−04 | 1.09E−08 |
| TRANS-2-BUTENE | 7.67E−05 | 6.15E−07 | 1.08E−05 | 1.56E−05 | 3.27E−05 | 3.75E−05 | 7.19E−05 |
| 1-HEXENE | 1.78E−03 | 9.35E−06 | 1.64E−04 | 5.98E−10 | 1.83E−04 | 1.89E−05 | 1.94E−03 |
| N-HEXANE | 3.59E−03 | 4.50E−05 | 7.83E−04 | 4.53E−11 | 1.16E−03 | 3.78E−04 | 4.37E−03 |
| 1-HEXADECENE | 9.94E−05 | 1.24E−09 | 2.05E−08 | 5.40E−10 | 2.97E−05 | 3.73E−10 | 1.29E−04 |
| N-METHYL-2- | 15.11605 | 3.15E−04 | 5.63E−03 | 3.89E−04 | 5.660847 | 3.20E−04 | 20.77658 |
| PYRROLIDONE | | | | | | | |
| Mole Frac | | | | | | | |
| HYDROGEN | 5.32E−04 | 0.169974 | 0.239676 | 0.209337 | 1.17E−04 | 0.019722 | 9.65E−27 |
| NITROGEN | 2.47E−05 | 7.95E−03 | 0.011223 | 9.80E−03 | 6.34E−06 | 9.29E−04 | 5.43E−29 |
| OXYGEN | 4.13E−05 | 5.96E−03 | 5.06E−03 | 4.70E−03 | 4.91E−04 | 7.82E−03 | 1.17E−20 |
| CARBON- | 2.12E−03 | 0.355234 | 0.377717 | 0.341948 | 4.25E−04 | 0.078051 | 1.86E−23 |
| MONOXIDE | | | | | | | |
| CARBON- | 1.30E−03 | 0.025302 | 0.018392 | 0.027904 | 1.37E−03 | 0.062322 | 2.21E−13 |
| DIOXIDE | | | | | | | |
| METHANE | 1.74E−03 | 0.162553 | 0.14537 | 0.140311 | 4.88E−04 | 0.065978 | 6.88E−21 |
| ACETYLENE | 7.09E−04 | 2.27E−03 | 1.57E−03 | 3.05E−07 | 0.02376 | 0.304927 | 6.30E−04 |
| ETHYLENE | 0.010212 | 0.255812 | 0.190157 | 0.258098 | 5.91E−03 | 0.426455 | 1.59E−13 |
| ETHANE | 1.35E−04 | 4.61E−03 | 3.52E−03 | 4.27E−03 | 6.69E−05 | 5.50E−03 | 8.03E−17 |
| PROPADIENE | 2.11E−05 | 9.31E−05 | 6.50E−05 | 2.27E−04 | 1.10E−03 | 0.014904 | 2.09E−06 |
| METHYL- | 1.53E−07 | 1.06E−06 | 7.49E−07 | 2.07E−06 | 2.32E−06 | 3.55E−05 | 8.29E−11 |
| ACETYLENE | | | | | | | |
| PROPYLENE | 1.48E−04 | 2.40E−03 | 1.74E−03 | 2.87E−03 | 2.13E−04 | 7.82E−03 | 7.89E−13 |
| PROPANE | 2.44E−06 | 6.40E−05 | 4.80E−05 | 6.39E−05 | 2.62E−06 | 1.18E−04 | 7.49E−17 |
| 1,3-BUTADIENE | 3.19E−04 | 9.46E−04 | 6.62E−04 | 1.24E−10 | 3.39E−04 | 2.37E−03 | 2.80E−04 |
| ISOBUTYLENE | 8.50E−06 | 6.68E−05 | 4.73E−05 | 1.20E−04 | 4.05E−05 | 8.19E−04 | 5.79E−10 |
| CIS-2-BUTENE | 6.62E−06 | 5.26E−05 | 3.66E−05 | 9.35E−05 | 2.51E−05 | 5.53E−04 | 5.25E−10 |
| TRANS-2-BUTENE | 4.98E−06 | 1.11E−05 | 7.73E−06 | 9.52E−06 | 5.57E−06 | 8.33E−05 | 3.46E−06 |
| 1-HEXENE | 1.16E−04 | 1.69E−04 | 1.18E−04 | 3.65E−10 | 3.13E−05 | 4.20E−05 | 9.34E−05 |
| N-HEXANE | 2.33E−04 | 8.14E−04 | 5.61E−04 | 2.77E−11 | 1.98E−04 | 8.40E−04 | 2.10E−04 |
| 1-HEXADECENE | 6.46E−06 | 2.24E−08 | 1.47E−08 | 3.30E−10 | 5.06E−06 | 8.29E−10 | 6.20E−06 |
| N-METHYL-2- | 0.982319 | 5.71E−03 | 4.03E−03 | 2.38E−04 | 0.965413 | 7.11E−04 | 0.998775 |
| PYRROLIDONE | | | | | | | |
| Stream (Example 3) | 226 | 224 | 327 | 328 | 329 | 330 | 318 |
| Substream: MIXED | | | | | | | |
| Mole Flow lbmol/hr | | | | | | | |
| HYDROGEN | 5.46E−26 | 1.46E−25 | 0 | 0 | 0 | 0 | 0 |
| NITROGEN | 3.07E−28 | 8.22E−28 | 0 | 0 | 0 | 0 | 0 |
| OXYGEN | 6.61E−20 | 1.77E−19 | 0 | 0 | 0 | 0 | 0 |
| CARBON- | 1.05E−22 | 2.82E−22 | 0 | 0 | 0 | 0 | 0 |
| MONOXIDE | | | | | | | |
| CARBON- | 1.25E−12 | 3.35E−12 | 1.06E−12 | 1.92E−13 | 3.26E−35 | 3.86E−23 | 0 |
| DIOXIDE | | | | | | | |
| METHANE | 3.90E−20 | 1.04E−19 | 0 | 0 | 0 | 0 | 0 |
| ACETYLENE | 3.57E−03 | 9.53E−03 | 1.45E−03 | 2.11E−03 | 1.12E−18 | 4.90E−09 | 0 |
| ETHYLENE | 9.02E−13 | 2.41E−12 | 8.16E−13 | 8.58E−14 | 2.43E−35 | 3.04E−23 | 0 |
| ETHANE | 4.55E−16 | 1.22E−15 | 0 | 0 | 0 | 0 | 0 |
| PROPADIENE | 1.18E−05 | 3.16E−05 | 5.43E−06 | 6.39E−06 | 1.59E−24 | 1.61E−13 | 0 |
| METHYL- | 4.69E−10 | 1.26E−09 | 2.46E−10 | 2.23E−10 | 2.61E−30 | 9.38E−19 | 0 |
| ACETYLENE | | | | | | | |
| PROPYLENE | 4.47E−12 | 1.20E−11 | 3.48E−12 | 9.88E−13 | 0.00E+00 | 2.90E−22 | 0 |
| PROPANE | 4.24E−16 | 1.13E−15 | 0 | 0 | 0 | 0 | 0 |
| 1,3-BUTADIENE | 1.59E−03 | 4.24E−03 | 4.79E−04 | 1.11E−03 | 2.26E−20 | 3.49E−10 | 0 |
| ISOBUTYLENE | 3.28E−09 | 8.77E−09 | 1.88E−09 | 1.39E−09 | 2.09E−29 | 7.10E−18 | 0 |
| CIS-2-BUTENE | 2.97E−09 | 7.95E−09 | 1.68E−09 | 1.30E−09 | 0 | 9.26E−18 | 0 |
| TRANS-2-BUTENE | 1.96E−05 | 5.23E−05 | 9.14E−06 | 1.04E−05 | 1.52E−17 | 2.73E−09 | 0 |
| 1-HEXENE | 5.29E−04 | 1.41E−03 | 6.82E−05 | 4.60E−04 | 2.31E−18 | 3.57E−09 | 0 |
| N-HEXANE | 1.19E−03 | 3.18E−03 | 2.77E−04 | 9.13E−04 | 2.07E−21 | 7.47E−11 | 0 |
| 1-HEXADECENE | 3.51E−05 | 9.39E−05 | 5.14E−12 | 2.54E−07 | 4.32E−07 | 2.97E−05 | 0 |
| N-METHYL-2- | 5.65437 | 15.12221 | 5.93E−06 | 0.05327 | 0.053048 | 5.65529 | 0.107233 |
| PYRROLIDONE | | | | | | | |
| Mole Frac | | | | | | | |
| HYDROGEN | 9.65E−27 | 9.65E−27 | 0 | 0 | 0 | 0 | 0 |
| NITROGEN | 5.43E−29 | 5.43E−29 | 0 | 0 | 0 | 0 | 0 |
| OXYGEN | 1.17E−20 | 1.17E−20 | 0 | 0 | 0 | 0 | 0 |
| CARBON- | 1.86E−23 | 1.86E−23 | 0 | 0 | 0 | 0 | 0 |
| MONOXIDE | | | | | | | |
| CARBON- | 2.21E−13 | 2.21E−13 | 4.61E−10 | 3.32E−12 | 6.15E−34 | 6.83E−24 | 0 |
| DIOXIDE | | | | | | | |
| METHANE | 6.88E−21 | 6.88E−21 | 0 | 0 | 0 | 0 | 0 |
| ACETYLENE | 6.30E−04 | 6.30E−04 | 0.632726 | 0.036474 | 2.11E−17 | 8.67E−10 | 0 |
| ETHYLENE | 1.59E−13 | 1.59E−13 | 3.55E−10 | 1.48E−12 | 4.59E−34 | 5.37E−24 | 0 |
| ETHANE | 8.03E−17 | 8.03E−17 | 0 | 0 | 0 | 0 | 0 |
| PROPADIENE | 2.09E−06 | 2.09E−06 | 2.36E−03 | 1.10E−04 | 2.99E−23 | 2.84E−14 | 0 |
| METHYL- | 8.29E−11 | 8.29E−11 | 1.07E−07 | 3.85E−09 | 4.92E−29 | 1.66E−19 | 0 |
| ACETYLENE | | | | | | | |
| PROPYLENE | 7.89E−13 | 7.89E−13 | 1.51E−09 | 1.71E−11 | 0.00E+00 | 5.13E−23 | 0 |
| PROPANE | 7.49E−17 | 7.49E−17 | 0 | 0 | 0 | 0 | 0 |
| 1,3-BUTADIENE | 2.80E−04 | 2.80E−04 | 0.208263 | 0.019152 | 4.26E−19 | 6.17E−11 | 0 |
| ISOBUTYLENE | 5.79E−10 | 5.79E−10 | 8.20E−07 | 2.41E−08 | 3.94E−28 | 1.25E−18 | 0 |
| CIS-2-BUTENE | 5.25E−10 | 5.25E−10 | 7.30E−07 | 2.24E−08 | 0 | 1.64E−18 | 0 |
| TRANS-2-BUTENE | 3.46E−06 | 3.46E−06 | 3.98E−03 | 1.80E−04 | 2.86E−16 | 4.82E−10 | 0 |
| 1-HEXENE | 9.34E−05 | 9.34E−05 | 0.029666 | 7.96E−03 | 4.36E−17 | 6.31E−10 | 0 |
| N-HEXANE | 2.10E−04 | 2.10E−04 | 0.120424 | 0.015777 | 3.90E−20 | 1.32E−11 | 0 |
| 1-HEXADECENE | 6.20E−06 | 6.20E−06 | 2.24E−09 | 4.38E−06 | 8.15E−06 | 5.24E−06 | 0 |
| N-METHYL-2- | 0.998775 | 0.998775 | 2.58E−03 | 0.920346 | 0.999992 | 0.999995 | 1 |
| PYRROLIDONE | | | | | | | |

| Stream (Example 3) | 200 | 201 | 202 | 203 | 204 | 207 | 209 | 208 |
|---|---|---|---|---|---|---|---|---|
| Mass Flow lb/hr | | | | | | | | |
| HYDROGEN | 7.721192 | 6.176954 | 1.544238 | 6.152601 | 0.024353 | 1.568591 | 0.655502 | 0.035428 |
| NITROGEN | 2.207047 | 1.765637 | 0.441409 | 1.75753 | 8.11E−03 | 0.449517 | 0.426539 | 0.022978 |
| OXYGEN | 0.517188 | 0.41375 | 0.103438 | 0.270916 | 0.142834 | 0.246272 | 0.215381 | 0.030891 |
| CARBON- | 75.73806 | 60.59045 | 15.14761 | 60.05606 | 0.534391 | 15.682 | 14.21873 | 1.463273 |
| MONOXIDE | | | | | | | | |
| CARBON- | 4.428842 | 3.543074 | 0.885768 | 2.418179 | 1.124894 | 2.010663 | 1.068323 | 0.94234 |
| DIOXIDE | | | | | | | | |
| METHANE | 16.74716 | 13.39772 | 3.349431 | 13.06168 | 0.336046 | 3.685477 | 3.111317 | 0.57416 |
| ACETYLENE | 11.21 | 8.967999 | 2.242 | 0.07335 | 9.142903 | 11.3849 | 0.053707 | 0.287384 |
| ETHYLENE | 1.00345 | 0.80276 | 0.20069 | 0.678905 | 0.123855 | 0.324545 | 7.050025 | 4.804875 |
| ETHANE | 0 | 0 | 0 | 0 | 0 | 0 | 0.140058 | 0.070306 |
| PROPADIENE | 0.207169 | 0.165735 | 0.041434 | 6.53E−04 | 0.166348 | 0.207781 | 3.43E−03 | 0.013195 |
| METHYL- | 1.70E−03 | 1.36E−03 | 3.40E−04 | 6.52E−07 | 1.36E−03 | 1.70E−03 | 3.96E−05 | 9.65E−05 |
| ACETYLENE | | | | | | | | |
| PROPYLENE | 0 | 0 | 0 | 5.03E−10 | 2.75E−15 | 2.75E−15 | 0.096882 | 0.101138 |
| PROPANE | 0 | 0 | 0 | 0 | 0 | 0 | 2.80E−03 | 1.81E−03 |
| 1,3-BUTADIENE | 0 | 0 | 0 | 0.056059 | 0.173536 | 0.173536 | 0.047144 | 0.268299 |
| ISOBUTYLENE | 0 | 0 | 0 | 4.18E−07 | 7.35E−08 | 7.35E−08 | 3.50E−03 | 7.54E−03 |
| CIS-2-BUTENE | 0 | 0 | 0 | 4.12E−07 | 3.41E−08 | 3.41E−08 | 2.71E−03 | 5.88E−03 |
| TRANS-2-BUTENE | 0 | 0 | 0 | 2.20E−03 | 7.37E−04 | 7.37E−04 | 5.71E−04 | 4.34E−03 |
| 1-HEXENE | 0.218195 | 0.174556 | 0.043639 | 0.01023 | 0.283312 | 0.326951 | 0.013046 | 0.15043 |
| N-HEXANE | 0 | 0 | 0 | 0.064851 | 0.209385 | 0.209385 | 0.063623 | 0.313153 |
| 1-HEXADECENE | 0 | 0 | 0 | 1.64E−07 | 0.021078 | 0.021078 | 4.33E−06 | 0.0223 |
| N-METHYL-2- | 0 | 0 | 0 | 0.052616 | 1499.051 | 1499.051 | 0.526517 | 1498.524 |
| PYRROLIDONE | | | | | | | | |
| Mass Frac | | | | | | | | |
| HYDROGEN | 0.064343 | 0.064343 | 0.064343 | 0.072678 | 1.61E−05 | 1.02E−03 | 0.023664 | 2.35E−05 |
| NITROGEN | 0.018392 | 0.018392 | 0.018392 | 0.020761 | 5.36E−06 | 2.93E−04 | 0.015399 | 1.52E−05 |
| OXYGEN | 4.31E−03 | 4.31E−03 | 4.31E−03 | 3.20E−03 | 9.45E−05 | 1.60E−04 | 7.78E−03 | 2.05E−05 |
| CARBON- | 0.631151 | 0.631151 | 0.631151 | 0.709414 | 3.54E−04 | 0.010214 | 0.513315 | 9.71E−04 |
| MONOXIDE | | | | | | | | |
| CARBON- | 0.036907 | 0.036907 | 0.036907 | 0.028565 | 7.44E−04 | 1.31E−03 | 0.038568 | 6.25E−04 |
| DIOXIDE | | | | | | | | |
| METHANE | 0.13956 | 0.13956 | 0.13956 | 0.154292 | 2.22E−04 | 2.40E−03 | 0.112323 | 3.81E−04 |
| ACETYLENE | 0.093417 | 0.093417 | 0.093417 | 8.66E−04 | 6.05E−03 | 7.42E−03 | 1.94E−03 | 1.91E−04 |
| ETHYLENE | 8.36E−03 | 8.36E−03 | 8.36E−03 | 8.02E−03 | 8.20E−05 | 2.11E−04 | 0.254515 | 3.19E−03 |
| ETHANE | 0 | 0 | 0 | 0 | 0 | 0 | 5.06E−03 | 4.66E−05 |
| PROPADIENE | 1.73E−03 | 1.73E−03 | 1.73E−03 | 7.72E−06 | 1.10E−04 | 1.35E−04 | 1.24E−04 | 8.75E−06 |
| METHYL- | 1.42E−05 | 1.42E−05 | 1.42E−05 | 7.70E−09 | 9.00E−07 | 1.11E−06 | 1.43E−06 | 6.40E−08 |
| ACETYLENE | | | | | | | | |
| PROPYLENE | 0 | 0 | 0 | 5.94E−12 | 1.82E−18 | 1.79E−18 | 3.50E−03 | 6.71E−05 |
| PROPANE | 0 | 0 | 0 | 0 | 0 | 0 | 1.01E−04 | 1.20E−06 |
| 1,3-BUTADIENE | 0 | 0 | 0 | 6.62E−04 | 1.15E−04 | 1.13E−04 | 1.70E−03 | 1.78E−04 |
| ISOBUTYLENE | 0 | 0 | 0 | 4.94E−09 | 4.86E−11 | 4.78E−11 | 1.26E−04 | 5.00E−06 |
| CIS-2-BUTENE | 0 | 0 | 0 | 4.87E−09 | 2.26E−11 | 2.22E−11 | 9.77E−05 | 3.90E−06 |
| TRANS-2-BUTENE | 0 | 0 | 0 | 2.60E−05 | 4.88E−07 | 4.80E−07 | 2.06E−05 | 2.88E−06 |
| 1-HEXENE | 1.82E−03 | 1.82E−03 | 1.82E−03 | 1.21E−04 | 1.87E−04 | 2.13E−04 | 4.71E−04 | 9.98E−05 |
| N-HEXANE | 0 | 0 | 0 | 7.66E−04 | 1.39E−04 | 1.36E−04 | 2.30E−03 | 2.08E−04 |
| 1-HEXADECENE | 0 | 0 | 0 | 1.94E−09 | 1.39E−05 | 1.37E−05 | 1.56E−07 | 1.48E−05 |
| N-METHYL-2- | 0 | 0 | 0 | 6.22E−04 | 0.991866 | 0.976362 | 0.019008 | 0.993951 |
| PYRROLIDONE | | | | | | | | |
| Total Flow lbmol/hr | 8.247701 | 6.59816 | 1.64954 | 6.165974 | 15.57294 | 17.22248 | 1.340637 | 15.44339 |
| Total Flow lb/hr | 120 | 96 | 24 | 84.65583 | 1511.344 | 1535.344 | 27.69984 | 1507.644 |
| Total Flow cuft/hr | 164.2334 | 131.3867 | 32.84669 | 259.3357 | 31.2737 | 115.982 | 40.11763 | 32.8099 |
| Temperature F. | 85 | 85 | 85 | 85.69314 | 91.04011 | 187.6262 | 229.7514 | 229.7514 |
| Pressure psia | 294.6959 | 294.6959 | 294.6959 | 139.6959 | 141.6959 | 140.6959 | 247.6959 | 247.6959 |
| Density lb/cuft | 0.730667 | 0.730667 | 0.730667 | 0.326433 | 48.32636 | 13.23778 | 0.690465 | 45.95089 |
| Average MW | 14.54951 | 14.54951 | 14.54951 | 13.72951 | 97.04935 | 89.14765 | 20.6617 | 97.62393 |

| Stream (Example 3) | 214 | 212 | 213 | 222 | 216 | 221 | 223 |
|---|---|---|---|---|---|---|---|
| Mass Flow lb/hr | | | | | | | |
| HYDROGEN | 0.016495 | 0.018933 | 0.674435 | 0.69093 | 1.38E−03 | 0.01788 | 4.05E−25 |
| NITROGEN | 0.010668 | 0.012311 | 0.438849 | 0.449517 | 1.04E−03 | 0.011709 | 3.16E−26 |
| OXYGEN | 0.020347 | 0.010544 | 0.225925 | 0.246272 | 0.092121 | 0.112468 | 7.77E−18 |
| CARBON- | 0.91347 | 0.549803 | 14.76853 | 15.682 | 0.069724 | 0.983194 | 1.08E−20 |
| MONOXIDE | | | | | | | |
| CARBON- | 0.880811 | 0.061529 | 1.129852 | 2.010663 | 0.352665 | 1.233476 | 2.02E−10 |
| DIOXIDE | | | | | | | |
| METHANE | 0.430066 | 0.144095 | 3.255411 | 3.685477 | 0.045946 | 0.476012 | 2.30E−18 |
| ACETYLENE | 0.284114 | 3.27E−03 | 0.056978 | 1.30E−05 | 3.627589 | 3.570624 | 0.341084 |
| ETHYLENE | 4.408336 | 0.396539 | 7.446564 | 11.8549 | 0.971964 | 5.3803 | 9.30E−11 |
| ETHANE | 0.062648 | 7.66E−03 | 0.147716 | 0.210364 | 0.011793 | 0.074441 | 5.02E−14 |
| PROPADIENE | 0.012989 | 2.06E−04 | 3.63E−03 | 0.014883 | 0.257292 | 0.268541 | 1.74E−03 |
| METHYL- | 9.42E−05 | 2.34E−06 | 4.19E−05 | 1.36E−04 | 5.45E−04 | 6.39E−04 | 6.91E−08 |
| ACETYLENE | | | | | | | |
| PROPYLENE | 0.095558 | 5.58E−03 | 0.102463 | 0.19802 | 0.052435 | 0.147993 | 6.91E−10 |
| PROPANE | 1.66E−03 | 1.56E−04 | 2.95E−03 | 4.61E−03 | 6.76E−04 | 2.33E−03 | 6.87E−14 |
| 1,3-BUTADIENE | 0.265472 | 2.83E−03 | 0.049972 | 1.10E−08 | 0.107638 | 0.057666 | 0.315444 |
| ISOBUTYLENE | 7.33E−03 | 2.07E−04 | 3.70E−03 | 0.011039 | 0.01333 | 0.020664 | 6.76E−07 |
| CIS-2-BUTENE | 5.72E−03 | 1.63E−04 | 2.87E−03 | 8.59E−03 | 8.24E−03 | 0.01396 | 6.13E−07 |
| TRANS-2-BUTENE | 4.30E−03 | 3.45E−05 | 6.05E−04 | 8.75E−04 | 1.83E−03 | 2.10E−03 | 4.03E−03 |
| 1-HEXENE | 0.149643 | 7.87E−04 | 0.013833 | 5.03E−08 | 0.015422 | 1.59E−03 | 0.163476 |
| N-HEXANE | 0.309276 | 3.88E−03 | 0.0675 | 3.90E−09 | 0.100061 | 0.032561 | 0.376776 |
| 1-HEXADECENE | 0.0223 | 2.78E−07 | 4.61E−06 | 1.21E−07 | 6.66E−03 | 8.37E−08 | 0.028959 |
| N-METHYL-2- | 1498.493 | 0.031261 | 0.557777 | 0.038585 | 561.1745 | 0.031698 | 2059.636 |
| PYRROLIDONE | | | | | | | |
| Mass Frac | | | | | | | |
| HYDROGEN | 1.09E−05 | 0.015149 | 0.023297 | 0.019681 | 2.44E−06 | 1.44E−03 | 1.96E−28 |
| NITROGEN | 7.08E−06 | 9.85E−03 | 0.015159 | 0.012804 | 1.84E−06 | 9.41E−04 | 1.53E−29 |
| OXYGEN | 1.35E−05 | 8.44E−03 | 7.80E−03 | 7.01E−03 | 1.62E−04 | 9.04E−03 | 3.77E−21 |
| CARBON- | 6.06E−04 | 0.439918 | 0.510146 | 0.446693 | 1.23E−04 | 0.079036 | 5.26E−24 |
| MONOXIDE | | | | | | | |
| CARBON- | 5.85E−04 | 0.049232 | 0.039028 | 0.057273 | 6.22E−04 | 0.099155 | 9.82E−14 |
| DIOXIDE | | | | | | | |
| METHANE | 2.85E−04 | 0.115296 | 0.112451 | 0.104979 | 8.10E−05 | 0.038265 | 1.11E−21 |
| ACETYLENE | 1.89E−04 | 2.62E−03 | 1.97E−03 | 3.70E−07 | 6.40E−03 | 0.287031 | 1.66E−04 |
| ETHYLENE | 2.93E−03 | 0.317286 | 0.257225 | 0.33768 | 1.71E−03 | 0.432505 | 4.51E−14 |
| ETHANE | 4.16E−05 | 6.13E−03 | 5.10E−03 | 5.99E−03 | 2.08E−05 | 5.98E−03 | 2.44E−17 |
| PROPADIENE | 8.62E−06 | 1.65E−04 | 1.26E−04 | 4.24E−04 | 4.54E−04 | 0.021587 | 8.44E−07 |
| METHYL- | 6.25E−08 | 1.88E−06 | 1.45E−06 | 3.87E−06 | 9.61E−07 | 5.14E−05 | 3.35E−11 |
| ACETYLENE | | | | | | | |
| PROPYLENE | 6.34E−05 | 4.46E−03 | 3.54E−03 | 5.64E−03 | 9.25E−05 | 0.011897 | 3.35E−13 |
| PROPANE | 1.10E−06 | 1.25E−04 | 1.02E−04 | 1.31E−04 | 1.19E−06 | 1.88E−04 | 3.34E−17 |
| 1,3-BUTADIENE | 1.76E−04 | 2.26E−03 | 1.73E−03 | 3.12E−10 | 1.90E−04 | 4.64E−03 | 1.53E−04 |
| ISOBUTYLENE | 4.87E−06 | 1.66E−04 | 1.28E−04 | 3.14E−04 | 2.35E−05 | 1.66E−03 | 3.28E−10 |
| CIS-2-BUTENE | 3.79E−06 | 1.31E−04 | 9.91E−05 | 2.45E−04 | 1.45E−05 | 1.12E−03 | 2.97E−10 |
| TRANS-2-BUTENE | 2.86E−06 | 2.76E−05 | 2.09E−05 | 2.49E−05 | 3.23E−06 | 1.69E−04 | 1.96E−06 |
| 1-HEXENE | 9.93E−05 | 6.30E−04 | 4.78E−04 | 1.43E−09 | 2.72E−05 | 1.28E−04 | 7.93E−05 |
| N-HEXANE | 2.05E−04 | 3.10E−03 | 2.33E−03 | 1.11E−10 | 1.77E−04 | 2.62E−03 | 1.83E−04 |
| 1-HEXADECENE | 1.48E−05 | 2.23E−07 | 1.59E−07 | 3.45E−09 | 1.17E−05 | 6.73E−09 | 1.41E−05 |
| N-METHYL-2- | 0.994755 | 0.025013 | 0.019267 | 1.10E−03 | 0.989878 | 2.55E−03 | 0.999402 |
| PYRROLIDONE | | | | | | | |
| Total Flow lbmol/hr | 15.38813 | 0.055255 | 1.395892 | 1.637279 | 5.863653 | 0.44972 | 20.80207 |
| Total Flow lb/hr | 1506.395 | 1.249784 | 28.94962 | 35.10687 | 566.9128 | 12.43985 | 2060.868 |
| Total Flow cuft/hr | 32.78545 | 2.480833 | 62.60631 | 192.613 | 11.76639 | 90.84726 | 50.60375 |
| Temperature F. | 230.2682 | 230.2682 | 227.9827 | 86.9777 | 96.35484 | 103.9764 | 456.6481 |
| Pressure psia | 164.6959 | 164.6959 | 164.6959 | 49.69595 | 51.69595 | 29.69595 | 31.69595 |
| Density lb/cuft | 45.94705 | 0.503776 | 0.462407 | 0.182266 | 48.18068 | 0.136932 | 40.72559 |
| Average MW | 97.89326 | 22.61838 | 20.73915 | 21.44221 | 96.68254 | 27.66131 | 99.07033 |
| Stream (Example 3) | 226 | 224 | 227 | 228 | 229 | 230 | 218 |
| Mass Flow lb/hr | | | | | | | |
| HYDROGEN | 1.10E−25 | 2.94E−25 | 0 | 0 | 0 | 0 | 0 |
| NITROGEN | 8.61E−27 | 2.30E−26 | 0 | 0 | 0 | 0 | 0 |
| OXYGEN | 2.11E−18 | 5.65E−18 | 0 | 0 | 0 | 0 | 0 |
| CARBON- | 2.95E−21 | 7.89E−21 | 0 | 0 | 0 | 0 | 0 |
| MONOXIDE | | | | | | | |
| CARBON- | 5.51E−11 | 1.47E−10 | 4.66E−11 | 8.45E−12 | 1.44E−33 | 1.70E−21 | 0 |
| DIOXIDE | | | | | | | |
| METHANE | 6.25E−19 | 1.67E−18 | 0 | 0 | 0 | 0 | 0 |
| ACETYLENE | 0.092827 | 0.248254 | 0.037857 | 0.05497 | 2.92E−17 | 1.28E−07 | 0 |
| ETHYLENE | 2.53E−11 | 6.77E−11 | 2.29E−11 | 2.41E−12 | 6.83E−34 | 8.53E−22 | 0 |
| ETHANE | 1.37E−14 | 3.66E−14 | 0 | 0 | 0 | 0 | 0 |
| PROPADIENE | 4.73E−04 | 1.27E−03 | 2.17E−04 | 2.56E−04 | 6.36E−23 | 6.44E−12 | 0 |
| METHYL- | 1.88E−08 | 5.03E−08 | 9.87E−09 | 8.94E−09 | 1.05E−28 | 3.76E−17 | 0 |
| ACETYLENE | | | | | | | |
| PROPYLENE | 1.88E−10 | 5.03E−10 | 1.46E−10 | 4.16E−11 | 0.00E+00 | 1.22E−20 | 0 |
| PROPANE | 1.87E−14 | 5.00E−14 | 0 | 0 | 0 | 0 | 0 |
| 1,3-BUTADIENE | 0.085848 | 0.229595 | 0.025886 | 0.059962 | 1.22E−18 | 1.89E−08 | 0 |
| ISOBUTYLENE | 1.84E−07 | 4.92E−07 | 1.06E−07 | 7.82E−08 | 1.17E−27 | 3.98E−16 | 0 |
| CIS-2-BUTENE | 1.67E−07 | 4.46E−07 | 9.41E−08 | 7.27E−08 | 0 | 5.20E−16 | 0 |
| TRANS-2-BUTENE | 1.10E−03 | 2.94E−03 | 5.13E−04 | 5.84E−04 | 8.52E−16 | 1.53E−07 | 0 |
| 1-HEXENE | 0.04449 | 0.118986 | 5.74E−03 | 0.038753 | 1.95E−16 | 3.00E−07 | 0 |
| N-HEXANE | 0.10254 | 0.274236 | 0.023847 | 0.078693 | 1.78E−19 | 6.44E−09 | 0 |
| 1-HEXADECENE | 7.88E−03 | 0.021078 | 1.15E−09 | 5.69E−05 | 9.70E−05 | 6.66E−03 | 0 |
| N-METHYL-2- | 560.5324 | 1499.104 | 5.88E−04 | 5.280755 | 5.258776 | 560.6236 | 10.63025 |
| PYRROLIDONE | | | | | | | |
| Mass Frac | | | | | | | |
| HYDROGEN | 1.96E−28 | 1.96E−28 | 0 | 0 | 0 | 0 | 0 |
| NITROGEN | 1.53E−29 | 1.53E−29 | 0 | 0 | 0 | 0 | 0 |
| OXYGEN | 3.77E−21 | 3.77E−21 | 0 | 0 | 0 | 0 | 0 |
| CARBON- | 5.26E−24 | 5.26E−24 | 0 | 0 | 0 | 0 | 0 |
| MONOXIDE | | | | | | | |
| CARBON- | 9.82E−14 | 9.82E−14 | 4.93E−10 | 1.53E−12 | 2.73E−34 | 3.03E−24 | 0 |
| DIOXIDE | | | | | | | |
| METHANE | 1.11E−21 | 1.11E−21 | 0 | 0 | 0 | 0 | 0 |
| ACETYLENE | 1.66E−04 | 1.66E−04 | 0.399988 | 9.97E−03 | 5.55E−18 | 2.28E−10 | 0 |
| ETHYLENE | 4.51E−14 | 4.51E−14 | 2.42E−10 | 4.37E−13 | 1.30E−34 | 1.52E−24 | 0 |
| ETHANE | 2.44E−17 | 2.44E−17 | 0 | 0 | 0 | 0 | 0 |
| PROPADIENE | 8.44E−07 | 8.44E−07 | 2.30E−03 | 4.64E−05 | 1.21E−23 | 1.15E−14 | 0 |
| METHYL- | 3.35E−11 | 3.35E−11 | 1.04E−07 | 1.62E−09 | 1.99E−29 | 6.70E−20 | 0 |
| ACETYLENE | | | | | | | |
| PROPYLENE | 3.35E−13 | 3.35E−13 | 1.55E−09 | 7.54E−12 | 0.00E+00 | 2.18E−23 | 0 |
| PROPANE | 3.34E−17 | 3.34E−17 | 0 | 0 | 0 | 0 | 0 |
| 1,3-BUTADIENE | 1.53E−04 | 1.53E−04 | 0.273507 | 0.010875 | 2.32E−19 | 3.37E−11 | 0 |
| ISOBUTYLENE | 3.28E−10 | 3.28E−10 | 1.12E−06 | 1.42E−08 | 2.23E−28 | 7.10E−19 | 0 |
| CIS-2-BUTENE | 2.97E−10 | 2.97E−10 | 9.94E−07 | 1.32E−08 | 0 | 9.27E−19 | 0 |
| TRANS-2-BUTENE | 1.96E−06 | 1.96E−06 | 5.42E−03 | 1.06E−04 | 1.62E−16 | 2.73E−10 | 0 |
| 1-HEXENE | 7.93E−05 | 7.93E−05 | 0.060617 | 7.03E−03 | 3.70E−17 | 5.36E−10 | 0 |
| N-HEXANE | 1.83E−04 | 1.83E−04 | 0.251961 | 0.014271 | 3.39E−20 | 1.15E−11 | 0 |
| 1-HEXADECENE | 1.41E−05 | 1.41E−05 | 1.22E−08 | 1.03E−05 | 1.84E−05 | 1.19E−05 | 0 |
| N-METHYL-2- | 0.999402 | 0.999402 | 6.21E−03 | 0.957694 | 0.999982 | 0.999988 | 1 |
| PYRROLIDONE | | | | | | | |
| Total Flow lbmol/hr | 5.661307 | 15.14076 | 2.30E−03 | 0.05788 | 0.053048 | 5.65532 | 0.107233 |
| Total Flow lb/hr | 560.8675 | 1500 | 0.094645 | 5.514031 | 5.258873 | 560.6302 | 10.63025 |
| Total Flow cuft/hr | 13.77187 | 36.83188 | 1.441946 | 0.116081 | 0.122277 | 13.002 | 0.218724 |
| Temperature F. | 456.6481 | 456.6481 | 110 | 110 | 371.0167 | 371.8612 | 85 |
| Pressure psia | 31.69595 | 31.69595 | 9.668387 | 9.668387 | 10.05512 | 214.6959 | 214.6959 |
| Density lb/cuft | 40.72559 | 40.72559 | 0.065637 | 47.50145 | 43.00776 | 43.11877 | 48.60109 |
| Average MW | 99.07033 | 99.07033 | 41.18834 | 95.26666 | 99.13362 | 99.13326 | 99.1326 |

TABLE 4

| | | Acetylene in | Ethylene |
|---|---|---|---|
| Sensitivity | C₂H₂ | Ethylene @100% | Recovery |
| of C₂H₂ | Fractional | Ethylene Basis | Efficiency |
| Conversion | Conversion | ppm | % |
| Example 1 | 0.930 | 19758 | 65.2 |
| | 0.940 | 16722 | 65.3 |
| | 0.950 | 13767 | 65.3 |
| | 0.960 | 10882 | 65.3 |
| | 0.970 | 8063 | 65.3 |
| | 0.980 | 5310 | 65.3 |
| | 0.990 | 2619 | 65.3 |
| | 0.995 | 1297 | 65.3 |
| | 0.999 | 250 | 65.3 |
| Example 2 | 0.930 | 19461 | 82.4 |
| | 0.940 | 16480 | 82.4 |
| | 0.950 | 13572 | 82.4 |
| | 0.960 | 10731 | 82.5 |
| | 0.970 | 7954 | 82.5 |
| | 0.980 | 5240 | 82.5 |
| | 0.990 | 2586 | 82.5 |
| | 0.995 | 1280 | 82.5 |
| | 0.999 | 247 | 82.5 |
| Example 3 | 0.930 | 1.42 | 100.0 |
| | 0.940 | 1.36 | 100.0 |
| | 0.950 | 1.30 | 100.0 |
| | 0.960 | 1.24 | 100.0 |
| | 0.970 | 1.18 | 100.0 |
| | 0.980 | 1.09 | 100.0 |
| | 0.990 | 0.72 | 100.0 |
| | 0.995 | 0.20 | 100.0 |
| | 0.999 | 0.02 | 100.0 |

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

Moreover, the scope of the present application is not intended to be limited to the particular embodiments of the process, machine, manufacture, composition of matter, means, methods and steps described in the specification.

1. A process for purifying and recovering ethylene from a cracked gas stream, the process comprising:
(a) hydrogenating a composition comprising acetylene and an acetylene extraction solvent in a hydrogenation unit under conditions sufficient to produce:
a first gaseous hydrocarbon stream comprising ethylene and acetylene; and
a first liquid stream comprising ethylene and acetylene gases dissolved in the acetylene extraction solvent,
wherein the first liquid stream comprises more acetylene than the first gaseous hydrocarbon stream;
(b) contacting the first gaseous hydrocarbon stream with a second acetylene extraction solvent stream in an acetylene scrubber unit under contacting conditions sufficient to extract a portion of the acetylene from the gaseous hydrocarbon stream and produce:
a second liquid stream comprising ethylene and acetylene gases dissolved in the acetylene extraction solvent, and
a gaseous ethylene product stream;
(c) contacting the first liquid stream of step (a) with the second liquid stream of step (b) in an ethylene stripper unit under separating conditions sufficient to produce a recovered gaseous ethylene stream and a third liquid stream comprising acetylene dissolved in the acetylene extraction solvent; and
(d) providing the recovered gaseous ethylene stream to step (b).

2. The process of claim 1, wherein the gaseous ethylene product stream comprises 1 ppm or less of acetylene, and/or the ethylene recovery efficiency is at least 80%, preferably 100%.

3. The process of claim 1, wherein the contacting conditions of step (b) and the separating conditions of step (c) comprise a pressure in the acetylene scrubber unit and a pressure in the ethylene stripper unit, and wherein the pressure in the ethylene stripper unit is less than the pressure in the acetylene scrubber unit.

4. The process of claim 3, wherein the acetylene scrubber unit pressure is from 0.01 MPa(g) to 0.5 MPa(g), preferably 0.03 MPa(g) to 0.5 MPa(g), with the ethylene stripper unit pressure preferably 0.01 MPa(g) to 0.4 MPa(g) less than the acetylene scrubber unit pressure.

5. The process of claim 1, wherein the acetylene is produced by partial oxidation of methane, methane pyrolysis, or heating a composition of methane and carbon oxides to produce a cracked gas stream comprising the acetylene.

6. The process of claim 5, further comprising separating a gaseous C₂+ hydrocarbons stream from the cracked gas stream.

7. The process of claim 6, further comprising contacting the C₂+ hydrocarbons stream with an acetylene extraction solvent to produce:
the liquid composition of step (a); and
acetylene.

8. The process of claim 7, wherein the acetylene extraction solvent comprises the third liquid stream of step (c).

9. The process of claim 1, further comprising cooling the third liquid stream to a temperature of 10° C. to 50° C.

10. The process of claim 1, further comprising:
separating in a regeneration column a portion of the third liquid stream of step (c) comprising the acetylene extraction solvent into a regenerated acetylene extraction solvent and a gaseous hydrocarbon stream comprising acetylene and light oligomers;
providing the regenerated acetylene extraction solvent to step (b); and
contacting in the scrubber column of step (b) the regenerated acetylene extraction solvent with the first gaseous hydrocarbon stream and the recovered ethylene stream from step (c).

11. The process of claim 10, further comprising cooling the regenerated acetylene extraction solvent prior to providing the regenerated acetylene extraction solvent to step (b).

12. The process of claim 1, further comprising:
flashing the first liquid stream of step (a) under reduced pressure, preferably 0.5 to 1.5 MPa(g) to produce:
a vapor stream that comprises ethylene and acetylene; and
a fourth liquid stream comprising the acetylene extraction solvent; and
combining the vapor stream with first gaseous hydrocarbon stream.

13. The process of claim 12, wherein the combined stream is cooled to about 20 to 30° C. prior to being provided to step (c).

14. The process of claim 1, wherein the fourth liquid stream is provided to the a lower section of the ethylene stripper unit of step (c).

15. The process of claim 1, further comprising removing the recovered gaseous ethylene stream from an upper section of the ethylene stripper unit and compressing the crude gaseous ethylene stream prior to step (d).

16. The process of claim 1, wherein hydrogenating step (a) comprises contacting the composition comprising acetylene and an acetylene extraction solvent with a hydrogenation catalyst, preferably a supported palladium and zinc catalyst.

17. The process of claim 1, wherein the hydrogenating conditions comprise a temperature of 90° C. to 125° C. and a pressure of 1.5 MPa(g) to 1.8 MPa(g).

18. The process of claim 1, further comprising preheating a hydrogenation feed mixture comprising a portion of the separated gaseous C₂+ hydrocarbons stream and the composition comprising acetylene and an acetylene extraction solvent to 90° C. to 125° C. prior to step (a).

19. The process of claim 1, wherein the acetylene extraction solvent is an organic amine, acetone, methanol, paraffinic or olefinic liquids, preferably dimethylformamide, most preferably, N-methyl, 2-pyrrolidone.

20. The process of claim 1, wherein the composition being hydrogenated in step (a) further comprises a sufficient amount of a cracked gas stream such that a H₂/acetylene ratio in the composition contacted in the hydrogenation unit of step (a) is 1.5:1 to 3:1.
